(19) United States
(12) Patent Application Publication
(10) Pub. No.: US 2001/0055764 A1
(43) Pub. Date: Dec. 27, 2001

(54) Microarray methods utilizing semiconductor nanocrystals

(76) Inventors: Stephen A. Empedocles, Mountain View, CA (US); Edith Y. Wong, Danville, CA (US); Vince E. Phillips, Sunnyvale, CA (US); R. Hugh Daniels, Palo Alto, CA (US)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/784,645

(22) Filed: Feb. 15, 2001

Related U.S. Application Data
(60) Provisional application No. 60/266,290, filed on Sep. 29, 2000.
(60) Provisional application No. 60/182,845, filed on Feb. 16, 2000.
(60) Provisional application No. 60/133,084, filed on May 7, 1999.

(51) Int. Cl. C12Q 1/68; G01N 33/537; G01N 33/543

(52) U.S. Cl. 435/6; 435/7.92

(57) ABSTRACT

The present invention provides a number of different methods for conducting assays with different types of addressable arrays utilizing semiconductor nanocrystals to enhance detection. The invention includes methods utilizing semiconductor nanocrystals with nucleic acid, protein and tissue arrays, for example. By utilizing various useful aspects of semiconductor nanocrystals, the invention also provides a variety of different options for conducting multiplexed assays. Additionally, detection methods involving counting of individual complexes that include semiconductor nanocrystals are provided which can be utilized to expand the dynamic range of detection.

### CROSS-REFERENCES TO RELATED APPLICATIONS

This application claims priority to U.S. Patent Application entitled “Microarray Methods Utilizing Semiconductor Nanocrystals,” filed Sep. 29, 2000, and having attorney docket number 019916-001200US, and claims the benefit of U.S. Provisional Patent Application No. 60/182,845, filed Feb. 16, 2000, both of which are incorporated by reference in their entirety for all purposes. This application is related to U.S. patent application Ser. No. 09/566,014, filed May 5, 2000, which claims the benefit of U.S. Provisional Application No. 60/133,084, filed May 7, 1999, both of which are also incorporated by reference in their entirety for all purposes.

### BACKGROUND OF THE INVENTION

Bioassays are used to probe the quantity of a target analyte present in a biological sample. Surface-based assays, in which the amount of target is quantified by capturing it on a solid support and then labeling it with a detectable label, are especially important since they allow for the facile separation of bound and unbound labels. Recently a number of surface-based assays have been developed that utilize different types of arrays.

Array-based assays are of importance because they permit a very large number of interrogations to be performed simultaneously by placing different “assays” on spatially distinct locations of an array. Addressable arrays can be fabricated to study many different analytes including proteins, DNA and RNA. In general, the sample to be tested is spread over the entire array so that target biomolecules in the sample can form complexes with their binding partner on the array. The target is typically labeled with some type of detectable tag (e.g., a fluorescent or radioactive label) so that the amount of each target analyte in the sample can be quantified by detecting the labeled complexes on the array.

One example of a surface based assay is a DNA microarray. The use of DNA microarrays has become widely adopted in the study of gene expression and genotyping due to the ability to monitor large numbers of genes simultaneously (see, e.g., Schena et al. (1995) Science 270:467-470; and Pollack et al. (1999) Nat. Genet. 23:41-46). More than 100,000 different probe sequences can be bound to distinct spatial locations across the microarray surface, each spot corresponding to a single gene (Schena et al (1998) Tibtech 16:301-306). When a fluorescently labeled DNA target sample is placed over the surface of the array, individual DNA strands hybridize to complementary strands within each array spot. The level of fluorescence detected quantifies the number of copies bound to the array surface and therefore the relative presence of each gene, while the location of each spot determines the gene identity. Using arrays, it is theoretically possible to simultaneously monitor the expression of all genes in an organism's genome. The use of DNA microarrays is an extremely powerful technique, with applications spanning all areas of genetics (see, e.g., the Chipping Forecast supplement to Nature Genetics 21 (1999)). Arrays can also be fabricated using other binding moieties such as antibodies, proteins, haptens or aptamers, in order to facilitate a wide variety of bioassays in array format.

Other surface based assays include microtiter plate-based ELISAs (enzyme-linked immunosorbent assays) in which the bottom of each well is coated with a different antibody. A protein sample is then added to each well along with a fluorescently labeled secondary antibody for each protein. Target proteins are captured on the surface of each well and secondarily labeled with a fluorophore. Fluorescence at the bottom of each well quantifies the amount of each target molecule in the sample. Similarly, antibodies or DNA can be bound to a microsphere such as a polymer bead and assayed as described above.

Often detection of binding complexes in array-based assays involves the detection of a fluorescently labeled species that is part of the binding complex. Currently, two fluorescent dyes predominate the field of addressable array assay techniques: cy3, having an emission peak at 565 nm, and cy5, having an emission peak at 670 nm. There are many properties of these dyes that are typical of most organic dyes and that can limit their use for providing quantitative results using addressable arrays. Fast photobleaching of the dyes is one problem. Photobleaching refers to the deterioration of fluorescence intensity upon prolonged and/or repeated exposure to excitation light. Photobleaching is dependent on the intensity of the excitation light and the duration of the illumination. Conversion of the dye into a nonfluorescent species is irreversible. Furthermore, photobleaching limits the amount of signal that can be collected from a given region of the microarray, thereby limiting discrimination of very low level signals. The chemical instability of cy5 makes this dye very unpredictable, and consequently makes it difficult to quantify accurately assay results obtained using this dye.

Low quantum yield is another shortcoming of organic dyes which limits the amount of light that can be collected. The broad emission spectra associated with organic dyes create overlap between different colored dyes. Such overlap requires complex deconvolution of the signal to quantify assay results, thereby limiting the dynamic range of the assay. While emission spectra are quite broad, excitation spectra of organic dyes tend to be quite narrow. Consequently, different wavelengths of light are required for excitation of each dye. Additionally, organic dyes have a small Stokes shift (the separation of the absorption and emission maxima) that can result in high autofluorescence and create problems with scattered excitation light, thereby increasing the signal background in these measurements.

Furthermore, with cy3 and cy5, a maximum of two colors can be used in detection. Consequently, it is difficult to perform analyses in a multiplex format in which multiple species are examined simultaneously in a single assay. For array-based assays, this limitation means that if multiple samples are to be tested for the same analytes multiple identical arrays must be used. For instance, if one seeks to determine the level of gene expression under 20 different conditions, it is necessary to run 20 different assays using 20 arrays, each with a comparison between a test condition and a reference condition. This technique requires more arrays and materials and is therefore very costly. It also introduces additional noise with each measurement since comparisons between different conditions are not made directly on the same array.

In addition to these problems associated with organic fluorescent dyes, limitations with regard to sensitivity and dynamic range is another problematic area in array-based assays. Dynamic range refers to the ability to simultaneously measure analyte over a wide range of concentrations. Using current detection technology, it is usually necessary to sacrifice linearity in the high concentration regime for detection sensitivity in the low concentration regime. This limits the dynamic range of a single experiment.

The performance of an assay is typically measured by its ability to specifically and quantitatively measure vanishingly small quantities of the target species under investigation. This is especially true for genetic analysis such as gene expression or genotyping, where the available quantity of genetic material is limited. For instance, using current detection technology with organic dye labels, gene expression analysis on DNA microarrays requires between 50 and 200 μg of total RNA for single array hybridization. This requires as many as 10⁵ cells (Duggan et al. (1999) Nature Genetics 21(n1 s): 10-14). In many instances, such as samples extracted through microdissection (Sgroi et al. (1999) Cancer Res. 59:5656-5661), these large quantities of material are simply not available. This greatly complicates the detection of such samples labeled with standard organic fluorophores.

Thus, there is a need to address the limitations associated with existing organic fluorescent dyes and to increase sensitivity and dynamic range in order to improve the results that can be obtained using surface-based arrays such as those conducted with addressable arrays.

### SUMMARY OF THE INVENTION

Methods for conducting a variety of array assays utilizing semiconductor nanocrystals as labels are provided herein. Various features of the semiconductor nanocrystals enhance signal detection relative to conventional organic dyes. For example, the semiconductor nanocrystals emit an intense signal that aids detection. In some instances, signals are sufficiently intense that a single semiconductor nanocrystal can be detected. By controlling the size and composition of the semiconductor nanocrystals, one can obtain semiconductor nanocrystals that emit at particular wavelengths. Further, while the semiconductor nanocrystals have large absorption cross sections, they have narrow, symmetric emission spectra. This means that a number of different semiconductor nanocrystals can be excited at a single wavelength but emit at a variety of distinct wavelengths. This feature is useful for assays conducted in multiplex formats. Because the semiconductor nanocrystals can be readily attached to a variety of different biomolecules, the semiconductor nanocrystals can be utilized in a variety of different microarray analyses. For example, the semiconductor nanocrystals can be utilized to label target molecules that are probed using nucleic acid arrays, protein arrays, tissue arrays or other arrays that utilize labeled targets and optical detection.

Accordingly, certain methods for detecting a ligand of interest in a sample involve initially providing a first plurality of antiligands immobilized on a solid support at positionally distinct locations thereon to provide a first array, wherein the plurality of antiligands comprises a first antiligand capable of binding specifically to a first ligand of interest. This array is then contacted with a sample containing or suspected of containing the first ligand, wherein the first ligand is linked through a linker to a first semiconductor nanocrystal before, during or after the contacting, under conditions in which the first ligand binds specifically to the first antiligand to form a first complex. Unbound ligand is optionally removed from the array. The location of the first complex is then identified by detecting, and optionally quantifying, the presence in the first complex of the first semiconductor nanocrystal.

Certain methods are used in analyzing variations in nucleic acids such as single nucleotide polymorphisms. Some of these methods involve providing a first plurality of nucleic acid primers having a 3′ end and a 5′ end and which primers are immobilized on a solid support at positionally distinct locations thereon to provide a first array, wherein the plurality of primers comprise a first primer complementary to a first target nucleic acid having an allelic site. The first array is then contacted with a sample containing or suspected of containing the first target nucleic acid, in the presence of a first terminating nucleotide linked to a first semiconductor nanocrystal through a linker, under conditions such that the first target nucleic acid hybridizes to the first primer to form a first target-primer complex and such that if the first terminating nucleotide is complementary to the nucleotide at the allelic site the first primer is extended to incorporate the first terminating nucleotide to provide an extended primer. The location or locations that includes extended primer is identified by detecting the presence therein of the first semiconductor nanocrystal.

Other methods are secondary interrogation or sandwich type assays. These methods typically involve providing a first plurality of antiligands immobilized on a solid support at positionally distinct locations thereon to provide a first array, wherein the first plurality of antiligands comprises a first antiligand that is a binding partner of a first ligand. The array is then contacted with a sample containing or suspected of containing the first ligand, whereby the first antiligand and the first ligand interact to form a first binary complex. The binary complex in turn is contacted with a second antiligand wherein the second antiligand is (i) a binding partner of the first ligand and (ii) linked to a first semiconductor nanocrystal through a linker, whereby the second antiligand binds to the first ligand in the first binary complex to form a first ternary complex. The location of the array that includes the first ternary complex is identified by detecting the presence therein of the first semiconductor nanocrystal.

Still other methods involve labeling a ligand after it has become bound to an array. Certain of these methods involve providing a first plurality of antiligands immobilized on a solid support at positionally distinct locations thereon to provide a first array, wherein the plurality comprises a first antiligand that is a binding partner of a first ligand. The first array is then contacted with a sample containing or suspected of the first ligand, whereby the first ligand and the first antiligand interact to form a first complex. The first ligand in the first complex is subsequently labeled with a first semiconductor nanocrystal. The location of the array that includes the first complex is identified by detecting the presence therein of the first semiconductor nanocrystal.

### BRIEF DESCRIPTION OF THE DRAWINGS

FIG. 1A is a graphical representation that depicts the results of a particular immunological assay involving a secondary interrogation of a complex between a capture antibody and a protein labeled with a semiconductor nanocrystal and a secondary antibody labeled with another semiconductor nanocrystal.

FIG. 1B is a graphical representation of a tertiary complex (capture antibody, protein labeled with semiconductor nanocrystal, secondary antibody labeled with another semiconductor nanocrystal) formed in a secondary interrogation according to one method of the invention.

FIGS. 2A-2B illustrate the optical properties associated with semiconductor nanocrystals as a consequence of the phenomenon of quantum confinement. FIGS. 2A and 2B show the absorption and emission spectra from different semiconductor nanocrystal samples, illustrating how the emission wavelength varies as a function of size. Absorption spectra have been normalized to the height of the first absorption peak and have been vertically offset for clarity. Inset numbers correspond to the average diameter of the quantum dots within each ensemble sample.

FIG. 2C illustrates how the material from which a semiconductor nanocrystal is constructed affects the wavelength at which it emits. Emission spectrum from semiconductor nanocrystals of three different materials are shown: CdSe (visible), InP (visible-near infrared) and InAs (infrared).

FIG. 3 provides a graph that illustrates photodegradation of semiconductor nanocrystals vs fluorescein under identical excitation conditions. Sample concentrations were matched (˜10-5 mol/l) and each was excited with ˜1 W/cm² of 488 nm light from an Ar+ laser. Note that while fluorescein photobleaches within the first few seconds, quantum dots actually increase slightly in intensity over the first minute.

FIGS. 4A and 4B illustrate single semiconductor nanocrystal detection.

FIG. 4A is a photograph of single semiconductor nanocrystals using a laser epifluorescence microscope. Each individual spot corresponds to the fluorescence from a single semiconductor nanocrystal.

FIG. 4B depicts spectra from single semiconductor nanocrystals. Wavelength is dispersed on the x-axis and position on the y-axis. Each horizontal line corresponds to the fluorescence spectrum from a single semiconductor nanocrystal. Note that different size semiconductor nanocrystals are easily identified by small changes in emission wavelength.

FIGS. 5A and 5B show a comparison between the absorption and emission spectra of fluorescein (FIG. 5A) and a comparable color semiconductor nanocrystal (FIG. 5B). Note that while the emission spectrum of the semiconductor nanocrystal is significantly narrower than that for fluorescein, the absorption spectrum extends far to the blue, allowing efficient excitation with all wavelengths shorter than the emission wavelength.

FIGS. 6A-6C illustrate the extension of dynamic range that can be achieved through single hybridization counting.

FIG. 6A is a graphic representation of the transition from the ensemble concentration regime to the single copy hybridization regime.

FIG. 6B is a graph showing simulated data demonstrating the improved sensitivity achieved through single hybridization detection.

FIG. 6C is a plot of the theoretical number of discrete points detected within a 100 μm diameter array spot as the total number of bound labels increases. The calculation assumes that individual labels cannot be distinguished if they reside within the same 0.5 μm diameter region and a random distribution of label locations with an average density that is uniform across the array spot. Saturation becomes significant above ˜6000 as the probability of finding 2 or more labels within the same diffraction limited spot increases.

FIG. 7 presents a schematic drawing of single quantum dot microscope.

FIGS. 8A-8E illustrate and summarize the steps in certain automated array scanning methods of the invention. Initially, sequential images are taken at periodic positions across the array (FIG. 8A). The array is then reconstructed (FIG. 8B). Pattern recognition is utilized to identify the location of the array spots relative to alignment spots (FIG. 8C). Within each spot the average intensity is measured as well as the total number of discrete points (FIG. 8D). Values for the average intensity and the total number of discrete points are exported (FIG. 8E).

### DETAILED DESCRIPTION

I. Definitions

As used in this specification and the appended claims, the singular forms “a,” “an” and “the” include plural references unless the content clearly dictates otherwise. Thus, for example, reference to “a semiconductor nanocrystal” includes a mixture of two or more such semiconductor nanocrystals, and an “analyte” includes more than one such analyte.

Unless defined otherwise, all technical and scientific terms used herein have the meaning commonly understood by a person skilled in the art to which this invention belongs. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991). As used herein, the following terms have the meanings ascribed to them unless specified otherwise.

The terms “semiconductor nanocrystal,” “quantum dot,” “Qdot™ nanocrystal” or simply “nanocrystal” are used interchangeably herein and refer to an inorganic crystallite between about 1 nm and about 1000 nm in diameter or any integer or fraction of an integer therebetween, generally between about 2 nm and about 50 nm or any integer or fraction of an integer therebetween, more typically about 2 nm to about 20 nm (such as 2, 3, 4, 5, 6, 7, 8, 9, 10, 11, 12, 13, 14, 15, 16, 17, 18, 19, or 20 nm). A semiconductor nanocrystal is capable of emitting electromagnetic radiation upon excitation (i.e., the semiconductor nanocrystal is luminescent) and includes a “core” of one or more first semiconductor materials, and may be surrounded by a “shell” of a second semiconductor material. A semiconductor nanocrystal core surrounded by a semiconductor shell is referred to as a “core/shell” semiconductor nanocrystal. The surrounding “shell” material typically has a bandgap energy that is larger than the bandgap energy of the core material and can be chosen to have an atomic spacing close to that of the “core” substrate. The core and/or the shell can be a semiconductor material including, but not limited to, those of the group II-VI (ZnS, ZnSe, ZnTe, CdS, CdSe, CdTe, HgS, HgSe, HgTe, MgS, MgSe, MgTe, CaS, CaSe, CaTe, SrS, SrSe, SrTe, BaS, BaSe, BaTe, and the like) and III-V (GaN, GaP, GaAs, GaSb, InN, InP, InAs, InSb, and the like) and IV (Ge, Si, and the like) materials, and an alloy or a mixture thereof.

A semiconductor nanocrystal is, optionally, surrounded by a “coat” of an organic capping agent. The organic capping agent can be any number of materials, but has an affinity for the semiconductor nanocrystal surface. In general, the capping agent can be an isolated organic molecule, a polymer (or a monomer for a polymerization reaction), an inorganic complex, and an extended crystalline structure. The coat is used to confer solubility, e.g., the ability to disperse a coated semiconductor nanocrystal homogeneously into a chosen solvent, functionality, binding properties, or the like. In addition, the coat can be used to tailor the optical properties of the semiconductor nanocrystal. Methods for producing capped semiconductor nanocrystals are discussed further below.

Thus, the terms “semiconductor nanocrystal,” “quantum dot” and “Qdot™ nanocrystal” as used herein denote a coated semiconductor nanocrystal core, as well as a core/shell semiconductor nanocrystal.

By “luminescence” is meant the process of emitting electromagnetic radiation (light) from an object. Luminescence results from a system which is “relaxing” from an excited state to a lower state with a corresponding release of energy in the form of a photon. These states can be electronic, vibronic, rotational, or any combination of the three. The transition responsible for luminescence can be stimulated through the release of energy stored in the system chemically or added to the system from an external source. The external source of energy can be of a variety of types including chemical, thermal, electrical, magnetic, electromagnetic, physical or any other type capable of causing a system to be excited into a state higher than the ground state. For example, a system can be excited by absorbing a photon of light, by being placed in an electrical field, or through a chemical oxidation-reduction reaction. The energy of the photons emitted during luminescence can be in a range from low-energy microwave radiation to high-energy x-ray radiation. Typically, luminescence refers to photons in the range from UV to IR radiation.

“Monodisperse particles” include a population of particles wherein at least about 60% of the particles in the population, more preferably 75% to 90% of the particles in the population, or any integer in between this range, fall within a specified particle size range. A population of monodispersed particles deviate less than 10% rms (root-mean-square) in diameter and typically less than 5% rms.

The phrase “one or more sizes of semiconductor nanocrystals” is used synonymously with the phrase “one or more particle size distributions of semiconductor nanocrystals.” One of ordinary skill in the art will realize that particular sizes of semiconductor nanocrystals are actually obtained as particle size distributions.

By use of the term “a narrow wavelength band” or “narrow spectral linewidth” with regard to the electromagnetic radiation emission of the semiconductor nanocrystal is meant a wavelength band of emissions not exceeding about 40 nm, and typically not exceeding about 20 nm in width and symmetric about the center, in contrast to the emission bandwidth of about 100 nm for a typical dye molecule with a red tail that can extend the bandwidth out as much as another 100 nm. It should be noted that the bandwidths referred to are determined from measurement of the full width of the emissions at half maximum peak height (FWHM), and are appropriate in the range of 200 nm to 2000 nm.

By use of the term “a broad wavelength band,” with regard to the excitation of the semiconductor nanocrystal is meant absorption of radiation having a wavelength equal to, or shorter than, the wavelength of the onset radiation (the onset radiation is understood to be the longest wavelength (lowest energy) radiation capable of being absorbed by the semiconductor nanocrystal). This onset occurs near to, but at slightly higher energy than the “narrow wavelength band” of the emission. This is in contrast to the “narrow absorption band” of dye molecules which occurs near the emission peak on the high energy side, but drops off rapidly away from that wavelength and is often negligible at wavelengths further than 100 nm from the emission.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms are used interchangeably. These terms refer only to the primary structure of the molecule. Thus, these terms include, for example, 3′-deoxy-2′,5′-DNA, oligodeoxyribonucleotide N3′P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels that are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine), those with intercalators (e.g., acridine, psoralen), those containing chelators (e.g., metals, radioactive metals, boron, oxidative metals), those containing alkylators, those with modified linkages (e.g., alpha anomeric nucleic acids), as well as unmodified forms of the polynucleotide or oligonucleotide. In particular, DNA is deoxyribonucleic acid.

The terms “polynucleotide analyte” and “nucleic acid analyte” are used interchangeably and include a single- or double-stranded nucleic acid molecule that contains a target nucleotide sequence. The analyte nucleic acids may be from a variety of sources, e.g., biological fluids or solids, chromosomes, food stuffs, environmental materials, etc., and may be prepared for the hybridization analysis by a variety of means, e.g., proteinase K/SDS, chaotropic salts, or the like.

As used herein, the term “target nucleic acid region” or “target nucleotide sequence” includes a probe-hybridizing region contained within the target molecule. The term “target nucleic acid sequence” includes a sequence with which a probe will form a stable hybrid under desired conditions.

As used herein, the term “nucleic acid probe” or simply “probe” includes reference to a structure comprised of a polynucleotide, as defined above, that contains a nucleic acid sequence complementary to a nucleic acid sequence present in the target nucleic acid analyte. The polynucleotide regions of probes may be composed of DNA, and/or RNA, and/or synthetic nucleotide analogs.

It will be appreciated that the hybridizing sequences need not have perfect complementarity to provide stable hybrids. In many situations, stable hybrids will form where fewer than about 10% of the bases are mismatches, ignoring loops of four or more nucleotides. Accordingly, as used herein the term “complementary” refers to an oligonucleotide that forms a stable duplex with its “complement” under assay conditions, generally where there is about 90% or greater homology.

An “array” broadly refers to an arrangement of antiligands in positionally distinct locations on a substrate. Typically the location of the antiligands on the array are spatially encoded so that the identity of an antiligand of an array can be deduced from its location on the array. A “microarray” generally refers to an array in which detection requires the use of microscopic detection to detect complexes formed between antiligands and ligands. A “location” on an array refers to a localized area on the array surface that includes antiligands, each defined so that it can be distinguished from adjacent locations (e.g., being positioned on the overall array or having some detectable characteristic that allows the location to be distinguished from other locations). Typically, each location includes a single type of antiligand. The location can have any convenient shape (e.g., circular, rectangular, elliptical or wedge-shaped). The size of an area can vary significantly. In some instances, the area of a location is greater than 1 cm², such as 2-20 cm², including any area within this range. More typically, the area of the location is less than 1 cm², in other instances less than 1 mm², in still other instances less than 0.5 mm², in yet still other instances less than 10,000 μm², or less than 100 μm².

A “solid support” includes planar or nonplanar substrates such as glass, nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

The term “aptamer” (or nucleic acid antibody) is used herein to refer to a single- or double-stranded DNA or a single-stranded RNA molecule that recognizes and binds to a desired target molecule by virtue of its shape. See, e.g., PCT Publication Nos. WO92/14843, WO91/19813, and WO92/05285, which are incorporated by reference herein.

The term “aptazyme” includes allosteric ribozymes that are activated in the presence of an effector molecule (either chemical or biological). Aptazymes are capable of transducing a noncovalent molecular recognition event into a catalytic event, for example, the production of a new covalent bond via ligation.

“Polypeptide” and “protein” are used interchangeably herein and include a molecular chain of amino acids linked through peptide bonds. The terms do not refer to a specific length of the product. Thus, “peptides,” “oligopeptides,” and “proteins” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide.

A “ligand” generally refers to any molecule that binds to an antiligand to form a ligand/antiligand pair. Thus, a ligand is any molecule for which there exists another molecule (i.e., the antiligand) that specifically binds to the ligand, owing to recognition of some portion or feature of the ligand.

An “antiligand” is a molecule that specifically or nonspecifically interacts with another molecule (i.e., the ligand).

A “target molecule” or “analyte” refers to the species whose presence, absence and/or concentration is being detected or assayed. In the array-based assays, described herein, the target molecule or analyte is also referred to as the ligand.

As used herein, the term “binding pair” or “binding partners” refers to first and second molecules that specifically bind to each other such as a ligand and an antiligand. The term binding pair or binding partners can refer to the antiligand and ligand that form a complex on an array. The terms can also refer to a first molecule attached to a ligand and a second molecule attached to a semiconductor nanocrystal that interact such that the ligand becomes attached to the semiconductor nanocrystal via the interacting binding pair members. “Specific binding” of the first member of the binding pair to the second member of the binding pair in a sample is evidenced by the binding of the first member to the second member, or vice versa, with greater affinity and specificity than to other components in the sample. The binding between the members of the binding pair is typically noncovalent. Binding partners need not necessarily be limited to pairs of single molecules. For example, a single ligand can be bound by the coordinated action of two or more antiligands. The result of binding between bind pairs or binding partners is a binding complex, sometimes referred to as a ligand/antiligand complex or simply as ligand/antiligand.

Exemplary binding pairs include: (a) any haptenic or antigenic compound in combination with a corresponding antibody or binding portion or fragment thereof (e.g., digoxigenin and anti-digoxigenin; fluorescein and anti-fluorescein; dinitrophenol and anti-dinitrophenol; bromodeoxyuridine and anti-bromodeoxyuridine; mouse immunoglobulin and goat anti-mouse immunoglobulin), (b) nonimmunological binding pairs (e.g., biotin-avidin, biotin-streptavidin, biotin-Neutravidin); (c) hormone [e.g., thyroxine and cortisol]-hormone binding protein; (d) receptor-receptor agonist or antagonist (e.g., acetylcholine receptor-acetylcholine or an analog thereof); (e) IgG-protein A (f) lectin-carbohydrate; (g) enzyme-enzyme cofactor; (h) enzyme-enzyme-inhibitor; (i) and complementary polynucleotide pairs capable of forming nucleic acid duplexes and the like.

The terms “specific-binding molecule” and “affinity molecule” are used interchangeably herein and refer to a molecule that will selectively bind, through chemical or physical means to a detectable substance present in a sample. By “selectively bind” is meant that the molecule binds preferentially to the target of interest or binds with greater affinity to the target than to other molecules. For example, an antibody will selectively bind to the antigen against which it was raised; A DNA molecule will bind to a substantially complementary sequence and not to unrelated sequences. The affinity molecule can comprise any molecule, or portion of any molecule, that is capable of being linked to a semiconductor nanocrystal and that, when so linked, is capable of recognizing specifically a detectable substance. Such affinity molecules include, by way of example, such classes of substances as antibodies, as defined below, monomeric or polymeric nucleic acids, aptamers, proteins, polysaccharides, sugars, and the like. See,. e.g., Haugland, “Handbook of Fluorescent Probes and Research Chemicals” (Sixth Edition), and any of the molecules capable of forming a binding pair as described above.

A “semiconductor nanocrystal conjugate” is a semiconductor nanocrystal that is linked to or associated with a specific-binding molecule, as defined above. A “semiconductor nanocrystal conjugate” includes, for example, a semiconductor nanocrystal linked or otherwise associated, through the coat, to a member of a “binding pair” or a “specific-binding molecule” that will selectively bind to a detectable substance present in a sample, e.g., a biological sample as defined herein. The first member of the binding pair linked to the semiconductor nanocrystal can comprise any molecule, or portion of any molecule, that is capable of being linked to a semiconductor nanocrystal and that, when so linked, is capable of recognizing specifically the second member of the binding pair.

The term “antibody” as used herein includes antibodies obtained from both polyclonal and monoclonal preparations, as well as, the following: (i) hybrid (chimeric) antibody molecules (see, for example, Winter et al. (1991) Nature 349:293-299; and U.S. Pat. No. 4,816,567); (ii) F(ab′)2 and F(ab) fragments; (iii) Fv molecules (noncovalent heterodimers, see, for example, Inbar et al. (1972) Proc Natl Acad Sci USA 69:2659-2662; and Ehrlich et al. (1980) Biochem 19:4091-4096); (iv) single-chain Fv molecules (sFv) (see, for example, Huston et al. (1988) Proc Natl Acad Sci USA 85:5879-5883); (v) dimeric and trimeric antibody fragment constructs; minibodies (see, e.g., Pack et al. (1992) Biochem 31:1579-1584; Cumber et al. (1992) J. Immunology 149B: 120-126); (vi) humanized antibody molecules (see, for example, Riechmann et al. (1988) Nature 332:323-327; Verhoeyan et al. (1988) Science 239:1534-1536; and U.K. Patent Publication No. GB 2,276,169, published Sep. 21, 1994); and, (vii) any functional fragments obtained from such molecules, wherein such fragments retain specific-binding properties of the parent antibody molecule.

Functional antibody fragments can be produced by cleaving a constant region, not responsible for antigen binding, from the antibody molecule, using e.g., pepsin, to produce F(ab′)2 fragments. These fragments contain two antigen binding sites, but lack a portion of the constant region from each of the heavy chains. Similarly, Fab fragments, comprising a single antigen binding site, can be produced, e.g., by digestion of polyclonal or monoclonal antibodies with papain. Functional fragments, including only the variable regions of the heavy and light chains, can also be produced, using standard techniques such as recombinant production or preferential proteolytic cleavage of immunoglobulin molecules. These fragments are known as Fv. See, e.g., Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single-chain Fv (“sFv” or “scFv”) polypeptide is a covalently linked VH-VL heterodimer which is expressed from a gene fusion including VH- and VL-encoding genes linked by a peptide-encoding linker. Huston et al. (1988) Proc. Natl. Acad. Sci. USA 85:5879-5883. A number of methods have been described to discern and develop chemical structures (linkers) for converting the naturally aggregated, but chemically separated, light and heavy polypeptide chains from an antibody V region into an sFv molecule which will fold into a three dimensional structure substantially similar to the structure of an antigen-binding site. See, e.g., U.S. Pat. Nos. 5,091,513, 5,132,405 and 4,946,778. The sFv molecules may be produced using methods described in the art. See, e.g., Huston et al. (1988) Proc. Nat. Acad. Sci. USA 85:5879-5883; U.S. Pat. Nos. 5,091,513, 5,132,405 and 4,946,778. Design criteria include determining the appropriate length to span the distance between the C-terminus of one chain and the N-terminus of the other, wherein the linker is generally formed from small hydrophilic amino acid residues that do not tend to coil or form secondary structures. Such methods have been described in the art. See, e.g., U.S. Pat. Nos. 5,091,513, 5,132,405 and 4,946,778. Suitable linkers generally comprise polypeptide chains of alternating sets of glycine and serine residues, and may include glutamic acid and lysine residues inserted to enhance solubility.

“Mini-antibodies” or “minibodies” are sFv polypeptide chains that include oligomerization domains at their C-termini, separated from the sFv by a hinge region. Pack et al. (1992) Biochem 31:1579-1584. The oligomerization domain comprises self-associating α-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein. Generally, minibodies are produced using recombinant methods well known in the art. See, e.g., Pack et al. (1992) Biochem 31:1579-1584; Cumber et al. (1992) J Immunology 149B:120-126.

As used herein, the term “monoclonal antibody” refers to an antibody composition having a homogeneous antibody population. The term is not limited regarding the species or source of the antibody, nor is it intended to be limited by the manner in which it is made. Thus, the term encompasses antibodies obtained from murine hybridomas, as well as human monoclonal antibodies obtained using human rather than murine hybridomas. See, e.g., Cote, et al. Monclonal Antibodies and Cancer Therapy, Alan R. Liss, 1985, p. 77.

A semiconductor nanocrystal is “linked” or “conjugated” to, or “associated” with, a specific-binding molecule or member of a binding pair when the semiconductor nanocrystal is chemically coupled to, or associated with the specific-binding molecule. Thus, these terms intend that the semiconductor nanocrystal can either be directly linked to the specific-binding molecule or can be linked via a linker moiety, such as via a chemical linker described below. The terms indicate species that are physically linked by, for example, covalent chemical bonds, physical forces such van der Waals or hydrophobic interactions, encapsulation, embedding, or the like. As an example without limiting the scope of the invention, semiconductor nanocrystals can be conjugated to molecules that can interact physically with biological compounds such as cells, proteins, nucleic acids, subcellular organelles and other subcellular components. For example, semiconductor nanocrystals can be associated with biotin which can bind to the proteins, avidin and streptavidin. Also, semiconductor nanocrystals can be associated with molecules that bind nonspecifically or sequence-specifically to nucleic acids (DNA, RNA). As examples without limiting the scope of the invention, such molecules include small molecules that bind to the minor groove of DNA (for reviews, see Geierstanger and Wemmer (1995) Ann. Rev. Biophys. Biomol. Struct. 24:463-493; and Baguley (1982) Mol. Cell. Biochem 43:167-181), small molecules that form adducts with DNA and RNA (e.g. CC-1065, see Henderson and Hurley (1996) J. Mol. Recognit. 9:75-87; aflatoxin, see Garner (1998) Mutat. Res. 402:67-75; cisplatin, see Leng and Brabec (1994) IARC Sci. Publ. 125:339-348), molecules that intercalate between the base pairs of DNA (e.g. methidium, propidium, ethidium, porphyrins; for a review see Bailly et al. J. Mol. Recognit. 5:155-171), radiomimetic DNA damaging agents such as bleomycin, neocarzinostatin and other enediynes (for a review, see Povirk (1996) Mutat. Res. 355:71-89), and metal complexes that bind and/or damage nucleic acids through oxidation (e.g. Cu-phenanthroline, see Perrin et al. (1996) Prog. Nucleic Acid Res. Mol. Biol. 52:123-151; Ru(II) and Os(II) complexes, see Moucheron et al. (1997) J. Photochem. Photobiol. B 40:91-106; chemical and photochemical probes of DNA, see Nielsen (1990) J. Mol. Recognit. 3:1-25.

As used herein, a “biological sample” refers to a sample of isolated cells, tissue or fluid, including but not limited to, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including, but not limited to, conditioned medium resulting from the growth of cells in cell culture medium, putatively virally infected cells, recombinant cells, and cell components).

A “small molecule” is defined as including an organic or inorganic compound either synthesized in the laboratory or found in nature. Typically, a small molecule is characterized in that it contains several carbon-carbon bonds, and has a molecular weight of less than 1500 grams/Mol.

A “biomolecule” is a synthetic or naturally occurring molecule, such as a protein, amino acid, nucleic acid, nucleotide, carbohydrate, sugar, lipid and the like.

The term “multiplexing” is used herein to include conducting an assay or other analytical method in which multiple analytes or biological states can be detected simultaneously by using more than one detectable label, each of which emits at a distinct wavelength, with a distinct intensity, with a distinct FWHM, with a distinct fluorescence lifetime, or any combination thereof. Preferably, each detectable label is linked to one of a plurality of first members of binding pairs each of which first members is capable of binding to a distinct corresponding second member of the binding pair. A multiplexed method using semiconductor nanocrystals having distinct emission spectra can be used to detect simultaneously in the range of 2 to 1,000,000, preferably in the range of 2 to 10,000, more preferably in the range of 2 to 100, or any integer between these ranges, and even more preferably in the range of up to 10 to 20, e.g., 2, 3, 4, 5, 6, 7, 8, 9, 10, 11, 12, 13, 14, 15, 16, 17, 18, 19, or 20, of analytes, biological compounds or biological states. Multiplexing also includes assays or methods in which the combination of more than one semiconductor nanocrystal having distinct emission spectra can be used to detect a single analyte.

“Optional” or “optionally” means that the subsequently described event or circumstance may or may not occur, and that the description includes instances where the event or circumstance occurs and instances in which it does not. For example, the phrase “optionally overcoated with a shell material” means that the overcoating referred to may or may not be present in order to fall within the scope of the invention, and that the description includes both the presence and absence of such overcoating.

A “site of variation,” “variant site” or “allelic site” when used with reference to a nucleic acid broadly refers to a site wherein the identity of nucleotide at the site varies between nucleic acids that otherwise have similar sequences. For double-stranded nucleic acids, the variant site includes the variable nucleotide on one strand and the complementary nucleotide on the other strand. A variant site can be the site of a single nucleotide polymorphism or the site of a somatic mutation, including a point mutation, a deletion, an insertion, and a rearrangement, for example.

“Polymorphism” refers to the occurrence of two or more alternative nucleotide sequences at a particular genetic locus in the genome of a population.

“Polymorphic form” or “allele” refers to alternative forms of a polymorphism that are exclusively distinguishable in an assay.

“Polymorphic marker” or “site” refers to a genetic locus at which divergence occurs. Preferred markers have at least two polymorphic forms, each occurring at frequency of greater than 1%, and more preferably greater than 10% or 20% of a selected population. A genetic locus can be as small as one base pair, if the polymorphism is a nucleotide substitution or deletion, or many base pairs if the polymorphism is, e.g., deletion, inversion or duplication of part of a chromosome. Polymorphic markers include, e.g., restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. One identified allelic form is arbitrarily designated as a the reference allele and other allelic forms are designated as alternative or variant alleles. The allelic form occurring most frequently in a selected population is sometimes referred to as the wild-type form. Diploid organisms may be homozygous or heterozygous for allelic forms. A di-allelic polymorphism has two forms. A tri-allelic polymorphism has three forms.

A single nucleotide polymorphism (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

A “primer” is a single-stranded polynucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically is at least 7 nucleotides long and, more typically range from 10 to 30 nucleotides in length. Short primer molecules generally require cooler temperatures to form sufficiently stable hybrid complexes with the template. A primer need not reflect the exact sequence of the template but must be sufficiently complementary to hybridize with a template. The term “primer site” or “primer binding site” refers to the segment of the target DNA to which a primer hybridizes. The term “primer pair” means a set of primers including a 5′ “upstream primer” that hybridizes with the complement of the 5′ end of the DNA sequence to be amplified and a 3′“downstream primer” that hybridizes with the 3′ end of the sequence to be amplified.

A primer that is “perfectly complementary” has a sequence fully complementary across the entire length of the primer and has no mismatches. The primer is typically perfectly complementary to a portion (subsequence) of a target sequence. A “mismatch” refers to a site at which the nucleotide in the primer and the nucleotide in the target nucleic acid with which it is aligned are not complementary. The term “substantially complementary” when used in reference to a primer means that a primer is not perfectly complementary to its target sequence; instead, the primer is only sufficiently complementary to hybridize selectively to its respective strand at the desired primerbinding site.

A “specimen” is a small part, or sample, of any substance or material obtained for analysis.

A “tissue” is an aggregation of similar cells united in the performance of a particular function. The four basic tissues are epithelium, connective tissues (including blood, bone and cartilage), muscle tissue and nerve tissue.

A “cellular specimen” is one that contains whole cells, and includes tissues. Examples include, but are not limited to, cells from the skin, breast, prostrate, blood, testis, ovary and endometrium.

A “cellular suspension” is a liquid in which cells are dispersed, and can include a uniform or non-uniform suspension. Examples of cellular suspensions are those obtained by fine-needle aspiration from tumor sites, cytology specimens , washes, urine that contains cells, ascitic fluid, or other bodily fluids.

A “cytological preparation” is a pathological specimen in which a cellular suspension can be converted into a smear or other form for pathological examination or analysis.”

A “tumor” is a neoplasm that may be either malignant or non-malignant. “Tumors of the same tissue type” refers to primary tumors originating in a particular organ (such as breast, prostrate, bladder or lung).

The term “naturally occurring” as applied to an object means that the object can be found in nature.

The term “subject” and “individual” are used interchangeably herein to refer to any type of organism, including, but not limited to, plants, animals and microorganisms.

II. Overview

The present invention provides a variety of methods for conducting assays with different types of addressable arrays using semiconductor nanocrystals (also referred to herein simply as a quantum dot or a Qdot™) as a label to enhance detection of various complexes formed on the array. Semiconductor nanocrystals can be used to label various ligands or target molecules for use in nucleic acid arrays, protein arrays, tissue arrays or essentially any other type of array that utilizes optical detection methods. The semiconductor nanocrystal labels can be directly incorporated into, or directly attached to, the ligands of interest through covalent or non-covalent attachment,or indirectly attached via a linker. By labeling ligands in different samples with different semiconductor nanocrystals, the methods can be used in multiplex formats to simultaneously evaluate a plurality of samples with a single array. Some methods also utilize ligands that each bear a single semiconductor nanocrystal.

By controlling various parameters, semiconductor nanocrystals utilized to label ligands or antiligands for use in array-based assays can be tailored to have a number of desired properties. For example, semiconductor nanocrystals can be produced that have characteristic spectral emissions. These spectral emissions can be tuned to a desired wavelength by varying the particle size, size distribution and/or composition of the particle. This means that multiple emission colors can be achieved, a feature that can be utilized in separately detecting ligands from different samples. The emission spectra of a population of semiconductor nanocrystals can be manipulated to have linewidths as narrow as 25-30 nm, depending on the size distribution heterogeneity of the sample population, and lineshapes that are symmetric, gaussian or nearly gaussian with an absence of a tailing region. The combination of tunability, narrow linewidths, and symmetric emission spectra enables high resolution of multiply sized semiconductor nanocrystals (e.g., populations of monodisperse semiconductor nanocrystals having multiple distinct size distributions within a system) and simultaneous detection of a variety of species.

In addition, the range of excitation wavelengths of such nanocrystals is broad and can be higher in energy than the emission wavelengths of all available semiconductor nanocrystals. This feature allows the use of a single energy source, such as light, usually in the ultraviolet or blue region of the spectrum, to effect simultaneous excitation of all populations of semiconductor nanocrystals in a system having distinct emission spectra. Semiconductor nanocrystals can also be more robust than conventional organic fluorescent dyes by having a high quantum yield, and typically are more resistant to photobleaching than the organic dyes conventionally utilized in array-based assays. The robustness of the nanocrystal also alleviates the problem of contamination of degradation products of the organic dyes in the system being examined. Moreover, semiconductor nanocrystals have a relatively large Stokes shift, thereby significantly reducing problems with autofluorescence and scattered excitation light. Therefore, arraybased technology used in combination with semiconductor nanocrystals can be used as a sensitive way to conduct a variety of assays and in certain instances the methods can be designed to allow for quantification of complexes formed on an array.

These various aspects of semiconductor nanocrystals also permit flexibility in methods for detecting and quantifying ligands as assayed using arrays. For example, the ability to detect single nanocrystals means that in some instances single ligands bound to the array can be individually counted. This capability means that one can quantitate the amount of ligand bound to the array, as well as quantifying the amount of ligand in the original sample containing the ligand by calibration against samples of known concentration.

The use of semiconductor nanocrystals also enables the dynamic range of detection to be extended relative to assays conducted with other types of labels. Depending upon the density of labeled ligand bound to the array, detection can involve counting of single ligands (lower densities) or determining the total emission intensity from each location of the array (higher ligand density). The ability to chose between these detection regimes results in significant expansion of the dynamic range of detection, thus allowing a greater range in the concentration of ligands that can accurately be quantified either as attached to the array or in the original sample.

III. Array-based Methods Utilizing Semiconductor Nanocrystals

A. General Methods

The present invention in general provides a variety of methods for assaying for ligands or target molecules using various array formats. Semiconductor nanocrystals are used as a labeling agent to enhance detection in several respects. The methods utilize arrays that include a substrate or support upon which a plurality of antiligands are placed or attached. If attached, the antiligands can be directly attached to the support, or attached via a linker. The array includes a variety of distinct locations to which the antiligands are placed or attached, hence the identity of the antiligands on the array is spatially encoded. Each location has at least one antiligand, but often there are a plurality of antiligands at each location. The antiligands at the various locations can be the same or different.

The array is contacted with a sample that contains, or potentially contains, one or more ligands. As the ligands in the sample are brought into contact with the antiligands of the array, ligands and antiligands that are members of a binding pair interact to form complexes. The ligands can be labeled with semiconductor nanocrystals either before or after the sample containing the ligands is contacted with the array.

The array is then typically rinsed to remove uncomplexed ligand and other assay components. Complexes formed on the array are identified by detecting a signal mediated by the semiconductor nanocrystals contained within the complexes. The identity of antiligands that have bound to a ligand can be determined based upon the location of the antiligand on the array.

Various modifications upon this general scheme can be made. For example, a sample containing one or more unlabeled ligands can be contacted with an array including multiple antiligands. As described above, ligands and antiligands that are binding partners form binary complexes. Since the ligands are unlabeled, complexes can be detected by contacting the binary complexes with a sample that contains secondary antiligands labeled with semiconductor nanocrystals. The secondary antiligands can bind to ligands in the binary complexes that are binding partners to form a tertiary complex. Those locations of the array in which an antiligand is complexed with a ligand can then be detected by a signal from the semiconductor nanocrystal in the tertiary complexes. This approach is a sandwich type assay in which the antiligand serves to capture a ligand which is its binding partner. The ligand is then bound to the labeled secondary antiligand such that the ligand is sandwiched between the two antiligands.

As described in greater detail infra, the ability to tune different semiconductors to emit at a distinctive wavelength by adjusting their size enables a variety of different multiplex analyses to be conducted. For example, different ligands from different samples can be separately labeled and then mixed together. The mixture can be applied to the array and complexes containing ligands from different samples identified on the basis of the color of the semiconductor nanocrystal within the complex. Alternatively, different ligands within a single sample can be differentially labeled by selectively attaching a first member of different binding pairs to the different ligands. The second member of the various binding pairs can than be selectively attached to different semiconductor nanocrystals. The resulting ligands and semiconductor nanocrystals can then be mixed. Different ligands within the sample become differentially labeled because each ligand only joins to a label that bears a complementary binding pair member.

B. Semiconductor Nanocrystals

Semiconductor nanocrystals are typically nanometer sized semiconductor crystals that have optical properties that are strongly dependent on both the size and the material of the crystal (see, e.g., Alivisatos (1996) Science 271:933-937). One feature of semiconductor nanocrystals is that the absorption and emission spectra from semiconductor nanocrystals can be tuned across a broad range of the electromagnetic spectrum by changing their size. For example, semiconductor nanocrystals manufactured from CdSe can emit light in a narrow wavelength band at any chosen wavelength between 490 nm and 640 nm.

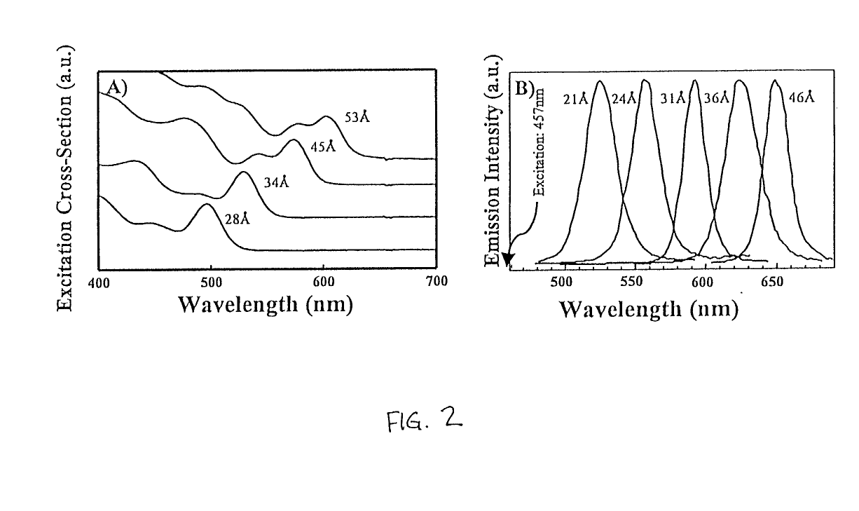

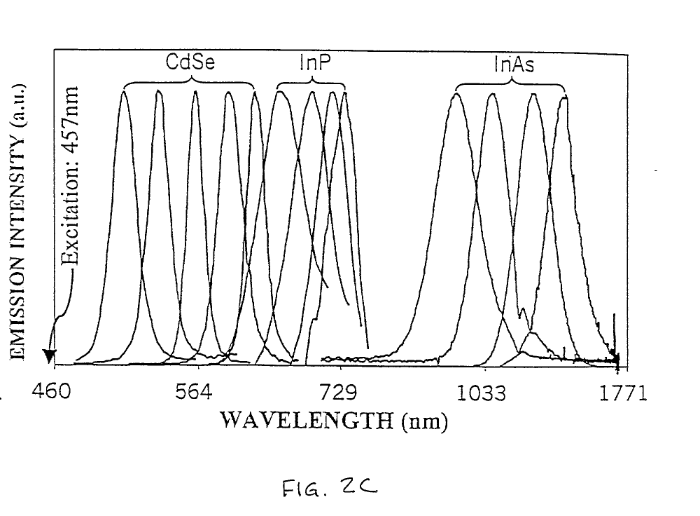

The principle behind the size dependent optical properties of semiconductor nanocrystals is an effect called “quantum confinement” (see, e.g., Efros, et al. (1982) Sov. Phys. Semicond. 16:772-775). Light emission from bulk semiconductors is generated through the creation and annihilation of an electron and anti-electron (hole) within the semiconductor lattice. In bulk semiconductors, the energy of this “electronhole pair” is governed entirely by the composition of the semiconductor material. If, however, the physical size of the semiconductor is reduced so that it is smaller than the intrinsic size of the electron-hole pair, additional energy is required to confine this excitation within the semiconductor structure. In the size range of semiconductor nanocrystals, the confinement energy can be extremely large, and becomes one of the dominant factors affecting the absorption and emission energies of the material. Therefore, by changing the size of the quantum dots, the absorption and emission can be modified due to changes in the confinement energy. FIGS. 2A-2B demonstrate this effect by showing a series of absorption and emission spectra from different size semiconductor nanocrystals of the same material (CdSe). Changing the material of the semiconductor nanocrystal can also affect the emission energy. By using a few different materials, it is possible to generate semiconductor nanocrystals with emission spectra that are tunable from the ultraviolet into the infrared (see FIG. 2C).

Semiconductor nanocrystals demonstrate quantum confinement effects in their luminescent properties. When semiconductor nanocrystals are illuminated with a primary energy source, a secondary emission of energy occurs at a frequency that corresponds to the bandgap of the semiconductor material used in the semiconductor nanocrystal. In quantum confined particles, the bandgap energy is a function of the size and/or composition of the nanocrystal. A mixed population of semiconductor nanocrystals of various sizes and/or compositions can be excited simultaneously using a single wavelength of light and the detectable luminescence can be engineered to occur at a plurality of wavelengths. The luminescent emission is related to the size and/or the composition of the constituent semiconductor nanocrystals of the population.

More specifically, quantum confinement of both the electron and hole in all three dimensions leads to an increase in the effective band gap of the material with decreasing crystallite size. Consequently, both the optical absorption and emission of semiconductor nanocrystals shift to the blue (higher energies). Upon exposure to a primary light source, each semiconductor nanocrystal distribution is capable of emitting energy in narrow spectral linewidths, as narrow as 12 nm to 60 nm full width of emissions at half peak height (FWHM), and with a symmetric, nearly Gaussian line shape, thus providing an easy way to identify a particular semiconductor nanocrystal. As one of ordinary skill in the art will recognize, the linewidths are dependent on, among other things, the size heterogeneity, i.e., monodispersity, of the semiconductor nanocrystals in each preparation. Certain single semiconductor nanocrystal complexes have been observed to have FWHM as narrow as 12 nm to 15 nm. Semiconductor nanocrystal distributions with larger linewidths in the range of 35 nm to 60 nm can be readily made and have the same physical characteristics as semiconductor nanocrystals with narrower linewidths.

Because the emission characteristics of semiconductor nanocrystals are dependent upon size and composition one can detect and/or distinguish between different semiconductor nanocrystals in a number of ways, including for example, emission intensity, emission wavelength, full width at half maximum peak height, absorption, scattering, fluorescence lifetime, or any combination of the foregoing.

A core/shell semiconductor nanocrystal is one made from one material such as CdSe that has been coated with a shell of a second, higher bandgap material such as ZnS (see, e.g., Hines et al. (1996) J. Phys. Chem. 100:468-471; Peng, et al. (1997) J. Am. Chem. Soc. 119:7019-7029; and Dabbousi, et al. (1997) J. Phys. Chem. B 101:9463-9475, each of which is incorporated by reference in its entirety). The higher bandgap shell material protects the fluorescent electron-hole pair from interacting with the surface and surrounding environment (such interactions can produce fluorescence quenching in semiconductor nanocrystals). This results in significantly enhanced fluorescence quantum yields, typically from 50% to 80%. These core/shell structures have a surface that is intrinsically functionalized with organic ligands.

As described further below, modification of these ligands allows one to make water soluble semiconductor nanocrystals that can be directly conjugated to biologically relevant molecules such as biotin, streptavidin and antibodies. Techniques for coupling semiconductor nanocrystals and a variety of biological molecules or substrates are described, for example, by Bruchez et. al (1998) Science 281:2013-2016, Chan et. al. (1998) Science 281:2016-2018, Bruchez “Luminescent Semiconductor Nanocrystals: Intermittent Behavior and use as Fluorescent Biological Probes” (1998) Doctoral dissertation, University of California, Berkeley, Mikulec “Semiconductor Nanocrystal Colloids: Manganese Doped Cadmium Selenide, (Core)Shell Composites for Biological Labeling, and Highly Fluorescent Cadmium Telluride” (1999) Doctoral dissertation, Massachusetts Institute of Technology, each of which is incorporated by reference in its entirety.

Several optical properties make semiconductor nanocrystals useful for detecting complexes in array-based methods. These properties include:

1) Large absorption cross sections. Semiconductor nanocrystals have very large absorption cross-sections relative to comparable organic dyes. For instance, Cy5 has a maximum cross section at approximately 630 nm of ˜250,000M⁻¹ cm⁻¹ while a red CdSe semiconductor nanocrystal (emission at 640 nm) has a cross section at 630 nm of ˜800,000 M⁻¹cm⁻³ and greater than 2×106 M⁻¹cm⁻1 at 488 nm. This means that with comparable excitation intensities at 488 nm and 632 nm for semiconductor nanocrystals and Cy5 respectively, semiconductor nanocrystals can absorb more than 8 times the amount of incident light.

2) High quantum yield. As described above, semiconductor nanocrystals can have quantum yields as high as 80%, hence a significant number of the absorbed photons are re-emitted as fluorescent signal.

3) High photostability. Unlike many organic dyes, semiconductor nanocrystals exhibit high photostability. FIG. 3 shows a comparison of photobleaching between fluorescein and water soluble semiconductor nanocrystals under identical excitation conditions. Enhanced photostability means that fluorescence from semiconductor nanocrystals can be integrated for long periods of time, thereby significantly enhancing detection sensitivity.

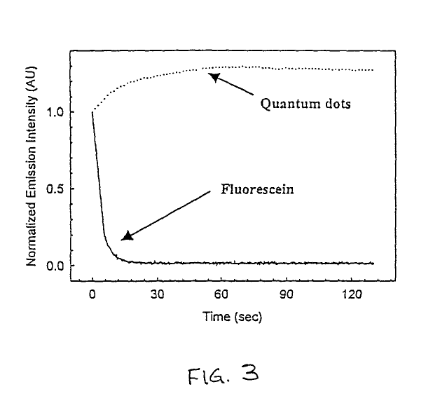

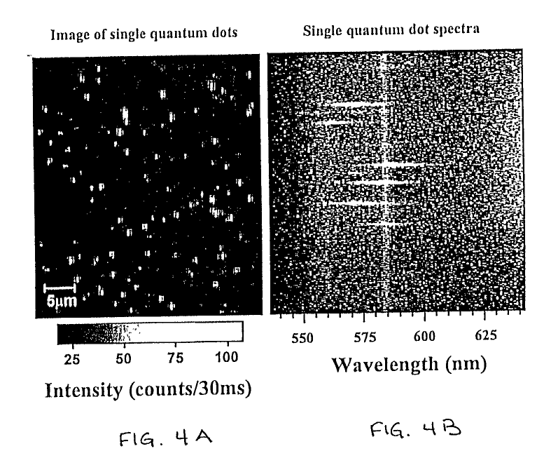

High absorption cross section, high quantum yield and high photostability combine to make an extremely bright fluorophore. With certain semiconductor nanocrystals, one can detect the fluorescence from single semiconductor nanocrystals (FIGS. 4A and 4B). In fact, the fluorescence from certain single semiconductor nanocrystals is sufficiently bright, that it can be seen by eye. This high fluorescence intensity can be exploited to allow the detection of single bound target molecules labeled with semiconductor nanocrystals as described in greater detail infra.

Additional useful characteristics of semiconductor nanocrystals include:

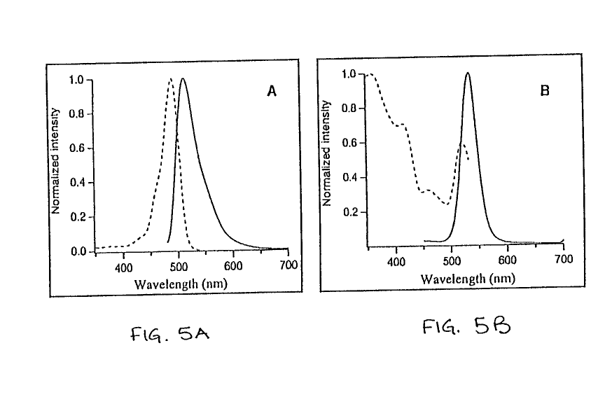

4) Narrow, symmetric emission spectra. The emission spectra from semiconductor nanocrystals are significantly narrower than most organic dyes, and do not have asymmetric tails extending to longer wavelengths. FIGS. 5A and 5B show a comparison between the absorption and emission spectra of fluorescein (FIG. 5A) and a comparable color semiconductor nanocrystal analogue (FIG. 5B). Narrow, symmetric emission spectra significantly reduce the overlap of adjacent colors in multiplexed assays, thereby increasing detection sensitivity.

5) Large “Stokes-shifts.” Contrary to the performance observed with organic dyes, shows that semiconductor nanocrystals actually absorb more light the farther away from the emission that they are excited (see FIGS. 5A and 5B). Hence, one can excite very far to the blue, minimizing any interference at the emission wavelength from scattered excitation light or autofluorescence, which generally results in emissions at wavelengths close to the excitation wavelength.

6) Tunable emission. Semiconductor nanocrystals can be synthesized to control the wavelength at which they emit. Emission wavelengths can therefore be selected to avoid overlap with autofluorescence. In addition, since semiconductor nanocrystals can also be excited at wavelengths shorter than the emission wavelength, excitation can also be chosen to avoid exciting autofluorescence. Appropriately chosen excitation and emission wavelengths can significantly reduce autofluorescence, thereby increasing detection sensitivity.

7) Multiplexible emission. Broad excitation spectra allow a large number of semiconductor nanocrystal colors to be simultaneously excited with a single excitation wavelength. For instance, all semiconductor nanocrystal samples in FIGS. 2A and 2B can be efficiently excited with 457 nm light. This characteristic is unique to semiconductor nanocrystals, and simplifies the development of optical systems for detection of multiplexed bioassays.

C. Labeling

A variety of methods are available for labeling biomolecules with semiconductor nanocrystals. In certain methods, the semiconductor nanocrystal and the biomolecule have appropriate functional groups that allow the two molecules to be coupled. Certain biomolecules can be labeled by labeling a component of the biomolecule (e.g., a monomer of a polymer) which becomes incorporated into the final biomolecule during synthesis (e.g., incorporation of a nucleotide labeled with a semiconductor nanocrystal into a nucleic acid). The semiconductor nanocrystal and biomolecule can also be linked via a linker. The linkers typically are bifunctional, having a functional group at each end. One end of the linker becomes attached to the semiconductor nanocrystal and the other end to the biomolecule.

Alternatively, the semiconductor nanocrystal can bear one member of a binding pair and the biomolecule the other member of the binding pair. The biomolecule and nanocrystal can thus be joined via the binding pair members.

Additional detail regarding labeling is set forth for the various types of array-based assays and in the conjugation section infra.

D. Arrays

As indicated above, an array broadly refers to an arrangement of biomolecules in positionally distinct locations on a substrate such that the identity of the various biomolecules in the array can be determined based upon their location in the array. The biomolecules of the array are attached to a support that maintains the relative position of the biomolecules of the array either directly or via a linker.

The support can be any material which can support a plurality of biomolecules and maintain the biomolecules such that they remain positionally distinct. Hence, the support can be manufactured from a wide variety of materials. For example, the support can be made of organic, inorganic, biological, or nonbiological materials or combinations of these materials. Specific examples of suitable supports include, but are not limited to, various plastics, polymers, Pyrexg, quartz, resins, silicon, silica or silica based materials, carbon, metals, inorganic glasses, inorganic crystals, cellulose, nylon and the like.

The form of the support can also vary. The support can have essentially any configuration. It may include a substantially planar surface or lack a planar surface. The substrate can have raised or depressed regions at which a reaction can occur or at which a solution or suspension can be placed. A specific example of such a support is a microtiter plate as is known in the art. Other suitable shapes for the support include, but are not limited to, beads, particles, strands, gels, sheets, membranes, tubing, capillaries, pads, films, plates and slides, for example. The array can also be in the form of a bundle of optical fibers, each fiber in the bundle having an end that is substantially planar or that includes a cavity etched into the end (see, e.g., U.S. Pat. No. 5,837,196 and PCT Publication WO 98/50782).

IV. Nucleic Acid Arrays

A. General Methods with Nucleic Acid Arrays

1. Contacting/Hybridization

A sample containing or potentially containing target nucleic acids from one or more sources is contacted with an array of nucleic acid probes attached at different locations on the array. The target nucleic acids are typically labeled with one or more semiconductor nanocrystals prior to contacting the array with a sample, or include a modified nucleotide that permits the facile labeling of the target nucleic acids after they have become hybridized to complementary probes attached to the array. For example, a modified nucleotide can be a nucleotide (e.g., dATP, dTTP, dGTP and dCTP) that has been functionalized with a group that reacts with a complementary functional group borne by the semiconductor nanocrystal. Alternatively, the modified nucleotide is attached to one member of a binding pair that specifically binds to the other member of the binding pair that is attached to a semiconductor nanocrystal.

After the target nucleic acids have been allowed to hybridize with complementary probes attached to the array, the array is optionally washed with a stringency buffer to remove unbound or non-specifically bound target nucleic acids. Hybridization complexes formed on the array are detected by detecting a signal associated or mediated by semiconductor nanocrystals attached to target nucleic acids that are within the hybridization complexes.

These series of steps can be automated utilizing various automated systems. These systems can include temperature controllers and mixers to regulate the reaction conditions as appropriate to the particular analysis being conducted. The systems can be programmable to program the temperature and mixing conditions, as well as to automatically dispense reagents, wash the array and perform detection assays. Information regarding such systems and components is described, for example, in PCT publication WO 95/3386, which is incorporated by reference in its entirety.

As just noted, the array typically is stringency washed following application of sample to the array to remove unbound target nucleic acids and to at least partially remove target nucleic acids that are not perfectly complementary to the probe nucleic acid to which they are bound. The stringency of selected hybridization conditions depends on various factors known in the art, including, e.g., temperature, ionic strength and pH. An extensive guide to the hybridization of nucleic acids is found in Tijssen, Techniques in Biochemistry and Molecular Biology—Hybridization with Nucleic Probe, “Overview of principles of hybridization and the strategy of nucleic acid assays” (1993), which is incorporated herein by reference.

Generally, “stringent conditions” are selected to be about 5-10° C. lower than the thermal melting point (Tm) for the specific sequence at a defined ionic strength pH. The Tm is the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Stringent conditions are those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of de-stabilizing agents such as formamide. For selective or specific hybridization, a positive signal is at least two times background, preferably 10 times background hybridization. “Moderately stringent hybridization conditions” include hybridization in a buffer of 40% formamide, 1 M NaCl, 1% SDS at 37° C., and a wash in 1× SSC at 45° C. A positive hybridization is at least twice background. Those of ordinary skill will readily recognize that alternative hybridization and wash conditions can be utilized to provide conditions of similar stringency.

2. Labeling of Nucleic Acids

A variety of methods can be utilized to incorporate semiconductor nanocrystals into the target nucleic acids to be analyzed by use of an array. One approach involves enzymatically incorporating semiconductor nanocrystal-dNTPs into reverse-transcribed cDNA by reverse transcribing a template nucleic acid in the presence of dNTPs labeled with semiconductor nanocrystals. In a second approach, the desired cDNA is amplified using polymerase chain reaction (PCR) primers labeled with one or more semiconductor nanocrystals.

Because the intensity of the emission of certain semiconductor nanocrystals is sufficiently high such that a single semiconductor nanocrystal can be detected, in some methods it is advantageous to incorporate just a single semiconductor into the nucleic acid. Incorporation of a single semiconductor nanocrystal into a nucleic acid can be achieved using primers that are labeled with a single semiconductor nanocrystal during amplification of target. As described in greater detail below, labeling with a single semiconductor nanocrystal allows one to quantify the amount of ligand (e.g., CDNA) that has hybridized to the array using the total fluorescence intensity. With existing organic dyes, low sensitivity requires that multiple fluorophores be attached to each cDNA for detection. This prevents a quantitative measure of cDNA hybridization, since the number of fluorophores/cDNA is not known.

A third nucleic acid labeling approach involves synthesizing cDNA from active group-functionalized dNTP using reverse transcriptase. The resulting unlabeled form is then labeled by directly conjugating the active groups to the surface of semiconductor nanocrystals. As used herein, an “active group” or “functional group” has means an atom or group of atoms that define the structure of a particular molecule or family of molecules and, at the same time, determines their properties. Exemplary functional groups include hydroxyl, sulfhydryl, carbonyl, carboxyl, amino and double or triple bonds. In one specific example of such an approach, an amine-functionalized dNTP is covalently bound to succinamidyl ester-functionalized semiconductor nanocrystals. By first generating the cDNA using unlabeled dNTPs and subsequently labeling the synthesized product, one can avoid the possibility of steric hindrance preventing transcription of template into cDNA.

Another option is to postpone labeling until after a target has become hybridized to a complementary probe nucleic acid borne by the array. For example, one can incorporate different active groups into different cDNA chains by reverse transcribing a template nucleic acid in the presence of different functionalized dNTPs. The synthesized cDNA is then hybridized to probes on the array, followed by conjugation of the semiconductor nanocrystals to the functional groups borne by the dNTPs that were incorporated into the synthesized cDNA. For example, four semiconductor nanocrystals each of a different color and each with a different surface functionalization can be washed over a hybridized array to label four different sets of cDNA. This approach has the advantage that the semiconductor nanocrystals are not present during the hybridization step, thereby minimizing potential interference by the nanocrystals with hybridization. This method also minimizes the possibility of cross-linking different cDNA strands during labeling if individual semiconductor nanocrystals have more than one functional group on the surface. This type of labeling can also be done for single color detection by using a single active group and conjugating after hybridization.

Another labeling approach is to fragment transcribed DNA and then end-label the fragments with a semiconductor nanocrystal-dTTP conjugate using terminal transferase.

In all of the above procedures, except those in which labeling occurs after hybridization, multiplexed assays can be performed by preparing and labeling different cDNA samples separately, and then blending the samples together prior to hybridization on the array.

Certain labeling option involve joining the target nucleic acid to a semiconductor nanocrystal via some type of linker. The linker can be any of a number of different homo- and hetero-bifunctional moieties that include a functional group at either end of a chain of molecules; the functional groups at each end can be the same or different. Examples of suitable linkers include, but are not limited to, straight or branched-chain carbon linkers, heterocyclic linkers and peptide linkers. Suitable linkers are available from Pierce Chemical Company in Rockford, Illinois and are described in EPA 188,256; U.S. Pat. Nos. 4,671,958; 4,659,839; 4,414,148; 4,669,784; 4,680,338, 4,569,789 and 4,589,071, and by Eggenweiler, H. M, (1998) Drug Discovery Today, 3: 552.

A number of labeling options involve the use of binding pair members, in which one member of the pair is attached to the semiconductor nanocrystal and the other member is attached to a nucleic acid or nucleotide incorporated therein. The binding pair members can be any set of molecules that specifically bind to one another. Suitable binding pair members include, but are not limited to, antigen/antibodies, biotin/streptavidin (or avidin or neutravidin) and oligosaccharide/lectin.

Specific examples of such labeling methods include the following. One option is to bind streptavidin-coated semiconductor nanocrystals to biotinylated cDNA. The reverse approach can also be taken in which biotin-coated semiconductor nanocrystals are bound to biotinylated cDNA through a streptavidin bridge. Another approach is to bind antibody-labeled semiconductor nanocrystals to antigen-labeled cDNA. For instance, digoxigenin-labeled cDNA can be bound to semiconductor nanocrystal-labeled anti-digoxigenin antibody. The member of the binding pair can be incorporated at internal locations within a cDNA by conducting the reverse transcription of a nucleic acid template in the presence of dNTPs labeled with the binding pair member(s). Alternatively, the binding pair member can be incorporated near the terminus of the target by using primers labeled with the binding pair member to amplify the target nucleic acid.

Target nucleic acids bearing multiple labels can be prepared by conducting transcription in the presence of dNTPs bearing a binding pair member or by amplifying the target with primers bearing multiple binding pairs. Target nucleic acids bearing a single semiconductor nanocrystal can be prepared by using primers bearing a single binding pair member to conduct amplification of the target nucleic acid. Furthermore, attachment of the semiconductor nanocrystals bearing a binding pair member to target nucleic acids bearing the other binding pair member can be done either before or after hybridization of target nucleic acids to probes on the arrays.

One can conduct multiplexing analyses using various multi-color approaches to distinguish between different samples. For example, one can synthesize different cDNAs in different reaction vessels by using dNTPs bearing different binding pair members for different samples. These cDNAs can then be labeled with different semiconductor nanocrystals in a single reaction vessel (or after hybridization to arrays) by adding semiconductors bearing different binding pair members that are complementary to the different binding pair members borne by the different target nucleic acids. Alternatively, target nucleic acids from different samples can be separately reacted with different semiconductors (conjugated to a binding pair member) in different reaction vessels. Even if the target nucleic acids bear the same binding pair member, target nucleic acids can be differentially labeled by adding different semiconductor nanocrystals into the different reaction vessels. The resulting labeled target nucleic acids can then be mixed together prior to applying the labeled target nucleic acids to the array.

For a detailed description of other methods for conjugating ligands such as nucleic acids to semiconductor nanocrystals, see, e.g., U.S. Pat. No. 5,990,479; Bruchez et. al. (1998) Science 281:2013-2016., Chan et. al. (1998) Science 281:2016-2018, Bruchez “Luminescent Semiconductor Nanocrystals: Intermittent Behavior and use as Fluorescent Biological Probes” (1998) Doctoral dissertation, University of California, Berkeley, and Mikulec “Semiconductor Nanocrystal Colloids: Manganese Doped Cadmium Selenide, (Core)Shell Composites for Biological Labeling, and Highly Fluorescent Cadmium Telluride” (1999) Doctoral dissertation, Massachusetts Institute of Technology, each of which is incorporated by reference in its entirety.

3. Array Synthesis

The arrays typically utilized when assaying target nucleic acids according to the methods of the present invention typically include some type of solid support to which nucleic acid probes are attached. The nucleic acid probes attached to the solid support are generally nucleic acids or uncharged nucleic acid analogs such as, for example, peptide nucleic acids that are disclosed in International Publication No. WO 92/20702; morpholino analogs that are described in U.S. Pat. Nos. 5,185,444, 5,034,506 and 5,142,047.

Nucleic acid arrays can be prepared in two general ways. One approach involves binding DNA from genomic or cDNA libraries to some type of solid support, such as glass for example. (See for example, Meier-Ewart, et al, Nature 361:375-376 (1993); Nguyen, C. et al., Genomics 29:207-216 (1995); Zhao, N. et al., Gene, 158:207-213 (1995); Takahashi, N., et al, Gene 164:219-227 (1995); Schena, et al., Science 270:467-470 (1995); Southern et al., Nature Genetics Supplement 21:5-9 (1999); and Cheung, et al., Nature Genetics Supplement 21:15-19 (1999), each of which is incorporated herein in its entirety for all purposes.)

The second general approach involves the synthesis of nucleic acid probes. One method involves synthesis of the probes according to standard automated techniques and then post-synthetic attachment of the probes to a support. See for example, Beaucage, Tetrahedron Lett., 22:1859-1862 (1981) and Needham-VanDevanter, et al., Nucleic Acids Res., 12:6159-6168 (1984), each of which is incorporated herein by reference in its entirety. A second category is the so-called “spatially directed” oligonucleotide synthesis approach. Methods falling within this category further include, by way of illustration and not limitation, light-directed oligonucleotide synthesis, microlithography, application by ink jet, microchannel deposition to specific locations and sequestration by physical barriers.

Light-directed combinatorial methods for preparing nucleic acid probes are described in U.S. Pat. Nos. 5,143,854 and 5,424,186 and 5,744,305; PCT patent publication Nos. WO 90/15070 and 92/10092; Fodor et al., Science 251:767-777 (1991); and Lipshutz, et al, Nature Genetics Supplement 21:20-24 (1999), each of which is incorporated herein by reference in its entirety. These methods combine solid-phase chemical synthesis and semiconductor-based lithography. Various masking strategies are utilized to reduce the number of synthesis cycles such as described in U.S. Pat. Nos. 5,571,639 and 5,593,839 to Hubbel et al., and by Fodor et al., Science 251:767-777 (1991), each of which is incorporated herein by reference in its entirety.

Other combinatorial methods which can be used to prepare arrays include spotting reagents on the support using ink jet printers (see, e.g., Pease et al., EP 728, 520, and Blanchard, et al. Biosensors and Bioelectronics II: 687-690 (1996), which are incorporated herein by reference in their entirety). Arrays can also be synthesized utilizing combinatorial chemistry by utilizing mechanically constrained flowpaths or microchannels to deliver monomers to cells of a support (see, e.g., Winkler et al., EP 624,059; WO 93/09668; and U.S. Pat. No. 5,885,837, each of which is incorporated herein by reference in its entirety).

4. Target Nucleic Acid Amplification

In some instances, the samples contain such a low level of target nucleic acids that it is useful to conduct a pre-amplification reaction to increase the concentration of the target nucleic acids. As described supra, amplification using primers or nucleotides labeled with semiconductor nanocrystals also provides a facile way to label the target nucleic acids of interest.

If samples are to be amplified, amplification is typically conducted using the polymerase chain reaction (PCR) according to known procedures. See generally, PCR Technology: Principles and Applications for DNA Amplification (H. A. Erlich, Ed.) Freeman Press, NY, N.Y. (1992); PCR Protocols: A Guide to Methods and Applications (Innis, et al., Eds.) Academic Press, San Diego, Calif. (1990); Mattila et al., Nucleic Acids Res. 19: 4967 (199 1); Eckert et al., PCR Methods and Applications 1: 17 (1991); PCR (McPherson et al. Ed.), IRL Press, Oxford; and U.S. Pat. Nos. 4,683,202 and 4,683,195, each of these being incorporated by reference in its entirety. Other suitable amplification methods include the ligase chain reaction (LCR) (see, e.g., Wu and Wallace, Genomics 4:560 (1989) and Landegren et al., Science 241:1077 (1988); transcription amplification (see, e.g., Kwoh et al., Proc. Natl. Acad. Sci. USA 86:1173 (1989)); self-sustained sequence replication (see, e.g., Guatelli et al., Proc. Natl. Acad. Sci. USA, 87:1874 (1990)); and nucleic acid based sequence amplification (NABSA) (see, e.g., Sooknanan, R. and Malek, L., Bio Technology 13: 563-65 (1995)), each of which are incorporated by reference in their entirety.

Further guidance regarding nucleic sample preparation is described in Sambrook, et al., Molecular Cloning: A Laboratory Manual, 2^{nd} Ed., Cold Spring Harbor Laboratory Press, (1989), which is incorporated herein by reference in its entirety.

B. Expression Arrays

1. General

Certain methods of the present invention involve the analysis of gene expression levels. In general terms, expression analysis involves the detection and quantification of mRNA levels (or cDNA derived therefrom) in one or more samples. In some instances, differently colored labels are used simultaneously to quantitate changes in gene expression levels, or to estimate changes in expression of one gene relative to another, in the cell under different conditions. For instance, cDNA prepared from a first specimen under one set of environmental conditions can be labeled with a first color tag and the cDNA derived from a second specimen, or the same first specimen under a different set of environmental conditions, can be labeled with a second color tag. Both samples are co-hybridized to the array and the differentially labeled first and second cDNA molecules compete to bind at each spot. The ratio of the two colors at each location gives a quantitative measure of the relative change in expression for the gene.

Expression analysis provides key insight into a variety of biological phenomenon. Cellular development and differentiation is one area in which expression analysis finds particular utility. In any given cell, only a fraction of all the encoded genes are expressed. The levels and timing of expression control cellular development, differentiation, function and physiology. Thus, monitoring gene expression can be used to analyze these processes. As an example, expression analysis can be utilized to assess differences in expression between different types of tissue. Expression studies can also provide important information into the genetic basis for aging and phenotypic differences.

Expression analysis can also be of value in studies of various diseases. For instance, expression analysis can be used to analyze the development and progression of cancer, since these events are accompanied by complex changes in the pattern of gene expression. Such studies can involve, for example, a comparison of gene expression in diseased tissue and normal tissue or infected tissue and normal tissue. Expression analysis can also be used in other clinical applications, including evaluating the effects of various drug treatments on expression. For instance, differences in gene expression for normal tissue treated with drugs or a drug candidate and normal tissue can be compared. Similar drug studies could be performed with diseased tissue and normal tissue. By determining which genes are expressed in various diseases, one can identify genes or their protein products that have potential utility as drugs or as drug targets. In other clinical applications, expression analysis can be used in toxicological evaluations by comparing expression levels between tissue treated with potential poisons or toxins and normal tissue. Of course, expression analysis can be used in a variety of other comparative studies to assess the impact of variations in gene expression.

2. Sample Preparation

a. RNA Isolation

Samples can be obtained from essentially any source from which nucleic acids can be obtained. Cells in the sample can be disrupted in a variety of ways to release the RNA therein (see for example, Watson, et al., Recombinant DNA, 2^{nd} Edition, Scientific American Books, NY 1992, which is incorporated herein by reference in its entirety). For example, nucleic acids may be released by mechanical disruption (such as repeated freeze/thaw cycles, abrasion and sonication), physical/chemical disruption, such as treatment with detergents (e.g., Triton, Tween, or sodium dodecylsulfate), osmotic shock, heat, or enzymatic lysis (e.g., lysosyme, proteinase K, and pepsin).

Once nucleic acids have been obtained, they typically are reversed transcribed into cDNA, although MRNA can be used directly. Following formation of the cDNA, generally sequences of interest are amplified according to any of the various amplification techniques that are known in the art such as those described infra. Labeled RNA can be prepared from a cDNA template using RNA polymerase, or methods known in the art.

3. Methods

Differential gene expression analysis in which gene expression under differing sets of conditions is monitored can be accomplished in two general ways. One approach, and the approach traditionally utilized, is to use multiple arrays. In this approach, a different array is utilized for each of the different samples, each sample corresponding to a different set of conditions. For example, as described above, one sample might contain nucleic acids obtained from a healthy cell, while a second sample contains nucleic acids from a diseased cell. In such an investigation, one array would be used to determine expression in the healthy cell and the other array used to determine expression in the diseased cell. The problem with this approach is that each measurement made for the different arrays has some error associated with it. By making comparisons between different arrays, the error in each measurement becomes cumulative, thereby increasing the total error. Semiconductor nanocrystals can be used to reduce error generated in this approach. For example, the nucleic acids from each sample can be labeled with one or more semiconductor nanocrystals according to any of the direct or indirect methods just described, either before or after hybridization of the nucleic acids to the array. Furthermore, such error reduction can be accomplished using samples individually labeled with detectably distinct semiconductor nanocrystals and by making simultaneous measurements with the same array.

However, because semiconductor nanocrystals can readily be tuned to emit at different wavelengths, one can simultaneously analyze numerous different samples on a single array by differentially labeling the target nucleic acids from different samples using the labeling methods set forth above. This ability with the use of semiconductors afford results in significant improvements in the noise level when comparing hybridization results from different expression conditions because the cumulative error associated with measurements for multiple arrays is avoided.

Details regarding methods for using arrays of nucleic acid probes for monitoring expression of mRNA molecules is set forth in PCT/US96/143839 and WO 97/17317, each of which is incorporated by reference in its entirety. With these methods the polynucleotides selected to hybridize with target polynucleotides are selected to be complementary to the mRNA targets of interest or amplification products therefrom. Additional discussion regarding the use of microarrays in expression analysis sufficient to guide one skilled in the art to conduct such analyses is set forth for example in Duggan, et al., Nature Genetics Supplement 21:10-14 (1999); Bowtell, Nature Genetics Supplement 21:25-32 (1999); Brown and Botstein, Nature Genetics Supplement 21:33-37 (1999); Cole et al., Nature Genetics Supplement 21:38-41 (1999); Debouck and Goodfellow, Nature Genetics Supplement 21:48-50 (1999); Bassett, Jr., et al., Nature Genetics Supplement 21:51-55 (1999); and Chakravarti, Nature Genetics Supplement 21:56-60 (1999), each of which is incorporated herein by reference in its entirety.

C. Genotyping/SNP Analysis

1. General

It has been found that relatively minor changes in the genome of an organism, including changes as small as a single nucleotide, can result in substantially different phenotypes. For example, these changes or mutations can be responsible for a variety of different diseases, influence the efficacy of different therapeutic treatments and alter the pathogencity of a microorganism or change the resistance of a microorganism to therapeutics directed towards it. Often such effects are the result of alteration of a single nucleotide. Such alterations are generally referred to as single nucleotide polymorphisms, or simply SNPs. The site at which an SNP occurs is referred to as a polymorphic site or an allelic site. A number of SNPs have been correlated with various human diseases (see, e.g., Publication WO 93/02216 which provides an extensive list of such SNPs). Because SNPs appear regularly throughout the genome, they also serve as useful genetic markers.

The ability to detect specific nucleotide alterations or mutations in DNA sequences has a number of medical and non-medical utilities. For example, methods capable of identifying nucleotide alterations provide a means for screening and diagnosing many common diseases that are associated with SNPs. Such methods are also valuable in identifying individuals susceptible to disease, those who could benefit from prophylactic measures, and thus obtaining information useful in patient counseling and education. Methods for detecting alterations and mutations have further value in the detection of microorganisms, and making correlations between the DNA in a particular sample and individuals having related DNA. This latter capability can be useful in resolving paternity disputes and in forensic analysis.

2. Methodology

General Considerations:

The invention provides a number of different methods for detecting one or more target nucleic acids having a particular sequence. In general, these methods involve providing an array that bears a plurality of nucleic acid probes having different sequences. Normally, probes of different sequence are positioned at different locations so that the identity of the probe is spatially encoded. A sample containing target nucleic acids labeled with semiconductor nanocrystals is contacted with the probe. Hybridization complexes between complementary probes and target nucleic acids is detected by detecting a signal associated with the semiconductor nanocrystal. As described further below, through the use of appropriate probes, one can detect the presence or absence of a particular target nucleic acid of interest.

Allele Specific Hybridization:

One method for detecting a target nucleic acid that has a particular polymorphic form is to utilize allele specific probes that each specifically hybridize with a particular polymorphic form of a target nucleic acid. By detecting which of the probes on the array form hybridization complexes, one can determine the presence or absence of particular target nucleic acids. Samples from different individuals can be probed on a single array by differentially labeling nucleic acids from the different individuals with different semiconductor nanocrystals. The labeled probes necessary for conducting the reaction can be prepared according to the methods set forth above, by synthesizing the probes with functionalized nucleotides that permit the post-synthetic attachment of the semiconductor nanocrystals or using one or more labeled nucleotides in the synthesis of the probes by standard methods.

The group of probes attached to the array support can include all the allelic probes that specifically hybridize to each of the different polymorphic forms of a target nucleic acid. Since most polymorphisms are biallelic, this means that the array includes two probes for each polymorphic form. If, however, the target nucleic acid is triallelic, then three probes each complementary to one of the three polymorphic forms can be utilized. Similarly, if the target nucleic acid is tetra-allelic, then the four probes complementary to the four different polymorphic forms can be included in the array. By labeling the target nucleic acids with semiconductor nanocrystals, detection of the hybridization complexes is enhanced; the ability to use different colored semiconductor nanocrystals means that samples from a number of individuals can be analyzed simultaneously.

Further guidance regarding allele-specific hybridization is set forth, for example, by Erlich, et al. (1991) Eur. J Immunogenet. 18:33-55; Zhang, et al. (1991) Nucleic Acids Res. 19:3929-3933; Impraim, et al. (1987) Biochem. Biophys. Res. Commun. 142:710-716; Saiki, et al. (1986) Nature 324:163-166; Wu, et al (1989) DNA 8:135-142; Thein, et al. (1988) Br. J. Haematol. 70:225-231; and Connor, et al. (1983) Proc. Natl. Acad. Sci. USA 80:278-282, and in PCT Publication 95/11995, each of which is incorporated by reference in its entirety.

Allele-Specific Ligation:

Other methods of the invention utilize semiconductor labeled probes to conduct allele-specific ligation reactions to detect the presence or absence of a particular target nucleic acid and distinguish between different polymorphic forms. In general, these methods involve contacting a target nucleic acid (which can be amplified prior to analysis) with a first probe that is complementary to a sequence adjacent the polymorphic site under hybridization conditions. This first probe hybridizes to a sequence that is possessed by all the target nucleic acids regardless of the nucleotide at the polymorphic site (i.e., regardless of the polymorphic form of the target). The target nucleic acid is also contacted with a second probe that is an allele-specific probe, i.e., a probe that only hybridizes to a particular polymorphic form of the target nucleic acid. The first and second probes are selected such that when the allele-specific probe is complementary with the nucleotide at the polymorphic site the two probes hybridize directly adjacent one another. In particular, the 3′ terminus of one probe is immediately adjacent the 5′ terminus of the other probe. So long as the allele-specific probe is complementary to the nucleotide at the polymorphic site, added ligase can join the two probes. By labeling either or both of the probes with semiconductor nanocrystals, detection of ligated product can be enhanced. The probes necessary for conducting these types of analyses can be prepared as described above in the section on allele-specific hybridization.

In some instances, one of the probes is attached to a solid support. In other methods, the ligation reaction is conducted in solution and the ligated products are detected after being captured by capture reagents attached to an array. In this latter instance, the capture reagents specifically recognize a tag attached to one or both of the probes. Different targets can be distinguished by different tags and/or by labeling different target nucleic acids with different semiconductor nanocrystals. These analyses can be conducted in multiplex format by differentially labeling the probes with different semiconductor nanocrystals. For example, the presence or absence of specific alleles can rapidly be determined by differentially labeling the allele-specific probes used to conduct the ligation reaction.

The general methods set forth above can be modified by conducting a series of ligase chain reactions or by coupling the ligase chain reaction with one or more PCR amplifications of the target nucleic acid as well. Further guidance on such methods is set forth, for example, in U.S. Pat. Nos. 5,830,711; 6,027,889; and 5,869,252, each of which are incorporated by reference in its entirety.

Primer Extension/Mini-Sequencing:

Certain methods of the invention involve conducting mini-sequencing reactions or primer extension reactions to identify the nucleotide present at a polymorphic site in a target nucleic acid. In general, in these methods a primer complementary to a segment of a target nucleic acid is extended if the reaction is conducted in the presence of a nucleotide that is complementary to the nucleotide at the polymorphic site. More specifically, the primer extension assays or mini-sequencing assays of the invention typically involve hybridizing a primer to a complementary target nucleic acid such that the 3′ end of the primer is immediately adjacent the polymorphic site or is a few bases upstream of the polymorphic site. The extension reaction is conducted in the presence of one or more nucleotides labeled with a semiconductor nanocrystal and a polymerase. Often the nucleotide is a dideoxynucleotide that prevents further extension by the polymerase once it is incorporated onto the 3′ end of the primer. If one of the added non-extendible nucleotides is complementary to the nucleotide at the polymorphic site, then a labeled nucleotide is incorporated onto the 3′ end of the primer to generate a labeled extension product. Because the incorporated nucleotide is complementary to the nucleotide at the polymorphic site, extended primers provide an indication of which nucleotide is present at the polymorphic site of target nucleic acids. Methods utilizing this general approach are discussed, for example, in U.S. Pat. Nos. 5,981,176; 5,846,710; 6,004,744; 5,888,819; 5,856,092; 5,710,028; and 6,013,431; and in PCT publication WO 92/16657, each of which is incorporated by reference.

In the array format of the present invention, the primers are typically attached to a support. These primers can be of random sequence or selected to be complementary to the target nucleic acids of interest. A sample containing target nucleic acids is contacted with the array of primers under conditions in which target nucleic acids become hybridized to complementary primers. Primers of the appropriate sequence hybridize to the target nucleic acid so that the 3′ end of the primer is adjacent to the polymorphic site of the target. Preferably, the 3′ end of the primer is immediately adjacent (but does not span) the polymorphic site (i.e., the 3′ end hybridizes to the nucleotide just upstream of the polymorphic site). If not already present, one or more nucleotides labeled with semiconductor nanocrystals are added. As indicated above, if the labeled nucleotides added include a nucleotide complementary to the nucleotide at the polymorphic site, the primer is extended by incorporation of a nucleotide bearing a semiconductor nanocrystal. By using four different semiconductor nanocrystals, each attached to a different nucleotide (e.g., ddATP, ddTTP, ddCTP and ddGTP), all possible alleles can be tested on a single array simultaneously.

In some instances methods can be performed with primers that simply hybridize adjacent to, but do not span, the polymorphic site. This is possible so long as none of the nucleotides on the target nucleic acid located between the 3′ end of the primer and the polymorphic site are the same as the nucleotide at the variant site. The extension reaction mixture in such instances must also include nucleotides complementary to those nucleotides positioned between the 3′ primer end and the polymorphic site.

In related methods, the primers include two general regions: a 5′ end region that includes a tag, and a 3′ region that is complementary to a target nucleic acid of interest. The array includes one or more capture reagents that can specifically bind with a tag borne by the primers. Typically, different capture reagents specific for different extension products are positioned at different locations on the array so that the identity of the capture reagents is spatially encoded. In general, the tag and capture reagent can be selected from any type of binding pairs in which the members of the pair specifically bind to one another. For example, the capture reagent can be a nucleic acid that is complementary to a nucleic acid segment of a primer (i.e., the primer tag). In methods of the invention utilizing this general approach, the extension reactions described above can be conducted in solution rather than on the array.

As set forth above, the reactions are conducted in the presence of one or more nucleotides (typically ddNTPs) labeled with a semiconductor nanocrystal. Following extension, the extension products in the extension reaction are contacted with the array. The capture reagents on the array capture primers bearing tags that are specifically recognized by the capture reagent. Those primers that have been extended can be detected by the semiconductor nanocrystal incorporated into the primer. The identity of the nucleotide at the polymorphic site of the target nucleic acid can be determined from the location on the array at which the extended product binds (see, e.g., U.S. Pat. No. 5,981,176).

In other methods for analyzing SNPs, an array of nucleic acid probes that are complementary to subsequences of a target sequence can be utilized to determine the identity of a target sequence, measure its amount, and detect differences between the target and a reference sequence using a procedure referred to as “tiling.” In brief, tiling strategies utilize a tiled array in which multiple nucleic acids that are identical except for one location are utilized. For example, in a 4L tiled array that typically is used for SNP analyses, there is a set of four probes of relatively short length (for example, 15-mers) which vary at the SNP position but which otherwise are identical and are perfect complements to a segment of the target nucleic acid being screened. A perfectly complementary probe binds more tightly to a target nucleic acid than those probes that have a single mismatch. Hence, the labeled probe generating the most intense signal corresponds to the probe having a nucleotide complementary to the nucleotide at the polymorphic site. The target nucleic acids can be labeled with semiconductor nanocrystals either before or after target nucleic acids have hybridized to the nucleic acid probes of the array.

The tiling approach to SNP analysis can be extended to examine long nucleic acid targets and detect numerous polymorphisms/mutations relative to a characterized consensus sequence. Additional guidance regarding such methods is generally available (see, e.g., WO 95/11995; U.S. Pat. No. 5,858,659; Chee, et al. Science 274:610-614 (1996); U.S. Pat. No. 5,837,832; and Lipshutz, et al., Bio Techniques 19:442-447 (1995), each of which is incorporated herein by reference in its entirety).

The labeled nucleotides utilized in the primer extension reactions can be prepared by directly attaching a semiconductor nanocrystal to the nucleotides via functional groups present on the naturally occurring nucleotides, or through different functional groups introduced onto the nucleotides. Alternatively, different nucleotides can bear different binding pair members (e.g., biotin or antibodies); the other complementary binding pair members are attached to the semiconductor nanocrystals. If functionalized nucleotides or nucleotides bearing binding pair members are used to conduct the extension reaction, then extension products can be labeled either before or after extension has occurred.

3. Target Nucleic Acid Preparation

The target nucleic acids utilized in SNP analyses can be extracted and isolated according to the methods generally described supra for expression analysis. If necessary, target nucleic acids can be amplified according to the various amplification methods also described above.

D. Sequencing Analysis

1. General

Traditional sequencing technologies involve complicated and time consuming procedures requiring electrophoretic size separation of labeled DNA fragments (see, e.g., Alphey, DNA Sequencing: From Experimental Methods to BioInformatics, Springer-Verlag, New York, 1997). An alternative approach involves using nucleic acid arrays to conduct hybridization studies with fragments of a target nucleic acid. From the hybridization results obtained, one can reconstruct the sequence of a target nucleic acid. In general, SBH uses a set of short nucleic acid probes of defined sequence to probe for complementary sequences on a longer target nucleic acid strand. The defined sequences that hybridize to the target can then be aligned using computer algorithms to construct the sequence of the target nucleic acid.

2. Methodology

The strategy of SBH can be illustrated by the following example. A 12-mer target DNA sequence, AGCCTAGCTGAA, is mixed with a complete set of octanucleotide probes. If only perfect complementarity is considered, five of the 65,536 octamer probes -TCGGATCG, CGGATCGA, GGATCGAC, GATCGACT, and ATCGACTT will hybridize to the target. Alignment of the overlapping sequences from the hybridizing probes reconstructs the complement of the original 12-mer target:

TCGGATCG

CGGATCGA

GGATCGAC

GATCGACT

ATCGACTT

TCGGATCGACTT

SBH can be performed in two formats. Hybridization methodology can be carried out by attaching target DNA to a surface. The target is then interrogated with a set of oligonucleotide probes, one at a time (see Strezoska et al., Proc. Natl. Acad. Sci. USA 88:10089-10093 (1991); and Drmanac et al., Science 260:1649-1652, (1993), each of which is incorporated by reference). Although this approach can be implemented with well established methods of immobilization and hybridization detection, it involves a large number of manipulations. For example, to probe a sequence utilizing a full set of octanucleotides, tens of thousands of hybridization reactions must be performed. In the second format, SBH is carried out by attaching probes to a surface in an array format where the identity of the probes at each site is known. Target nucleic acid, typically fragmented, is then added to the array of probes. The hybridization pattern determined in a single experiment can directly reveal the identity of all complementary probes.

The methods of the invention utilize target nucleic acids labeled with semiconductor nanocrystals using either of these two formats. Most typically, however, the latter approach is utilized. Hence, in certain methods, sequencing begins with the fragmenting of the target nucleic acid into fragments using various techniques known in the art (e.g., the use of restriction enzymes, or heating in the presence of high salt concentrations). The resulting fragments are labeled with semiconductor nanocrystals, diluted in buffer and then applied to an array bearing nucleic acid probes. The fragments are allowed to hybridize to the probes, typically using an automated apparatus to control temperature and sample mixing. The array is then optionally rinsed with a stringency buffer to remove unbound fragments and hybridization complexes detected by detecting a signal from the semiconductor nanocrystal used to label the fragments. The target nucleic acid can be labeled with semiconductor nanocrystals using any of the various methods described supra in the section on expression analysis.

Variations of the SBH procedure have been developed, primarily to address a problem with SBH, namely the problem of mismatches creating errors in the sequence determination. One such method termed “positional SBH” (PSBH) utilizes duplex probes having 3′ single-stranded overhangs to capture the target, and is followed by enzymatic ligation of the target to the duplex probe. This approach is designed to reduce mismatches (see for example, Broude, et al., Proc. Natl. Acad. Sci. USA 91:3072-3076 (1994); and U.S. Pat. No. 5,631,134 to Cantor, both of which are incorporated by reference in their entirety). PSBH itself has been further modified so that following the ligation reaction, DNA polymerase is added to extend the immobilized probe as a way of further reducing mismatches during capture of the target (see, e.g., Kuppuswamy, et al, Proc. Natl. Acad. Sci. USA 86:1143-1147 (1991), which is incorporated by reference in its entirety). The target nucleic acid fragments, probes or nucleotides used in such approaches can be labeled with semiconductor nanocrystals to enhance detection.

Additional guidance regarding sequencing by hybridization is provided, for example ,by Lysov et al., Dokl. Akad. Nauk SSSR 303:1508-1511 (1988); Bains et al, J. Theor. Biol. 135:303-307 (1988); Dimanac et al., Genomics 4:114-128 (1989); Barinaga, Science 253:1489 (1991); Stresoska et al., Proc. Natl. Acad. Sci. USA 88:10089 (1992); Bains, BioTechnology 10:757-758 (1992); and U.S. Pat. No. 5,202,231, each of which is incorporated by reference in its entirety.

V. Aptamers

Aptamers are single- or double-stranded DNA or single-stranded RNA molecules that recognize and bind to a desired target molecule by virtue of their shapes. See, e.g., PCT Publication Nos. WO92/14843, WO91/19813, and WO92/05285. The SELEX procedure, described in U.S. Pat. No. 5,270,163 to Gold et al., Tuerk et al. (1990) Science 249:505-510, Szostak et al. (1990) Nature 346:818-822 and Joyce (1989) Gene 82:83-87, can be used to select for RNA or DNA aptamers that are target-specific.

In certain methods, an oligonucleotide pool is constructed wherein an n-mer, typically a randomized sequence of nucleotides thereby forming a “randomer pool” of oligonucleotides, is flanked by two polymerase chain reaction (PCR) primers. The oligonucleotides in the pool are labeled with semiconductor nanocrystals according the methods described above. The array utilized in the screening of aptamers, typically bears the target molecules (e.g., proteins or small molecules) to be screened. The array is then contacted with the oligonucleotide pool under conditions that favor binding of the oligonucleotides to the target molecules on the array. Those oligonucleotides that bind the target molecule are separated from non-binding oligonucleotide using stringency washes. Those oligonucleotides that bind to the target molecules on the array are dissociated from the array using known techniques and then amplified using conventional PCR technology to form a ligand-enriched pool of oligonucleotides. Further rounds of binding, separation, dissociation and amplification are performed until an aptamer with the desired binding affinity, specificity or both is achieved. The final aptamer sequence identified can then be prepared chemically or by in vitro transcription.

VI. Protein Arrays

A. General

Protein arrays can be used to investigate interactions between proteins and a wide variety of different types of molecules such as nucleic acids, various small molecules and other proteins, for example. Protein arrays can be designed according to the aims of the particular investigation. For instance, an array can contain all the combinatorial variants of a bioactive protein or specific variants of a single protein species (e.g., splice variants, domains, or mutants), a family of protein orthologs from different species, a protein pathway, or even the entire protein complement of an organism. The arrays can also include antibodies, recombinant proteins, purified proteins and receptors, for example.

Protein arrays can be assigned to two general types. One type is referred to as a nonliving or chemical array. These protein arrays are composed of synthetic proteins. Arrays of this type are useful to investigate specific interactions between relatively small proteins with other proteins, particular nucleic acids or metal ions, for example. The second type is the biological or living protein array. These arrays include living entities that express proteins, including, but not limited to, viruses or cells. The arrays can include pools of proteins, cell fractions or intact cells. This type of array can be used to investigate more complex biological activities, including, but not limited to, activities involving multicomponent complexes or multistep enzymatic or signaling pathways. Regardless of type, the proteins typically are placed at positionally distinct locations on the array so that the different proteins are spatially encoded.

Certain features of semiconductor nanocrystals make them useful in protein arrays. For example, tunability permits multicolor simultaneous detection and, hence, multiple sample detection. There is also no need for enzyme development as with certain traditional ELISA methods. The semiconductor nanocrystals have increased photostability relative to organic fluorophores, thereby increasing detection sensitivity by virtue of the ability to monitor the signal over a long period of time.

B. Array Preparation

A variety of options are available for synthesizing proteins for use in nonliving arrays. One approach is to utilize various solid state synthesis approaches to synthesize the proteins on a desired support. For example, a method for synthesizing an array of peptides in microtiter wells has been described (see, e.g., Geysen, H. M. et al. (1984) Proc. Natl. Acad. Sci. USA 81:3998-4002. Other solid state methods are discussed, for example, by Merrifield, J. Am. Chem. Soc. (1963) 85:2149-2154; Atherton, Solid Phase Peptide Synthesis, IRL Press, Oxford (1989); Erickson, B. W. and Merrifield, R. B. (1976) in The Proteins, (Neurath, H. and Hill, R. L., eds.) Academic Press, New York, vol. 2, pp. 255-527; and Meienhofer, J. (1973) in Hormonal Proteins and Peptides, (Li, C.H., ed.) Academic Press, New York, vol. 2, pp. 45-267, each of which is incorporated by reference in its entirety.

A technique sometimes referred to as “spot synthesis” has also been developed using chemistry similar to the solid state methods. This particular approach takes advantage of the abundant hydroxyl groups present on cellulose filters to derivatize Fmoc-β-alanine groups. Peptide arrays can then be synthesized via the cellulose-bound alanine following deprotection (see, e.g., Gausepohl, H., et al., Pept. Res. 5:315-320 (1992); and Kramer, A. and Schneider-Mergener, J., Methods in Molecular Biology 87:25-39 (1998)). Other methods can be used to synthesize proteins on polymeric rods using solid state chemistry (see, e.g., Geysen, et al., Proc. Natl. Acad. Sci. USA 81:3998-4002 (1984)).

Methods allowing for the preparation and purification in parallel of large numbers of recombinant proteins can also be utilized to generate proteins for arrays. Such methods are discussed, for example, by Martzen, M. R., et al., Science 286:1153-1155 (1999).

Proteins can be synthesized directly on a solid support using various photolithographic methods. These techniques allow the number of proteins synthesized per unit area to be greatly increased. Examples of such methods are discussed, for example, in U.S. Pat. Nos. 5,143,854; 5,489,678; and 5,744,305; PCT Publications WO 90/15070 and WO 92/10092; and by Fodor, S. P. et al., Science 251:767-773 (1991), each of which is incorporated by reference in its entirety.

Instead of synthesizing the proteins on an array, preformed proteins can be directly deposited on a support to form the arrays. Thus, for example, recombinant proteins, purified proteins and the like can be blotted onto a support.

The proteins synthesized for use in non-living protein arrays need not be limited to proteins composed of the L-amino acids. Other building blocks, such as D-amino acids and modified amino acids can also be used.

Living arrays can also be prepared utilizing different methods. As indicated above, the arrays can be formed from pools of proteins, cellular extracts or intact cells. The pools, extracts or intact cells typically are placed in some type of support having a series of depressions (e.g., a microtiter plate) to contain the proteins. Methods for producing genome-wide protein arrays have also been described. Certain of these methods involves transformation events in which one of the open-reading frames (ORF) from an organism (e.g., yeast) is inserted into plasmid encoding for glutathione-S-transferase (GST). This plasmid is subsequently used to transform the organism which then expresses different GST-ORF fusion proteins. The resulting transformed cell cultures or colonies can be used as an element of an array (i.e., different colonies or groups of colonies are placed at different locations of the array; see, e.g., Martzen, M. R., et al., (1999)Science 286:1153-1155).

C. General Assays

1. Background

Protein arrays can be used to assay or screen for a variety of different types of activity or to conduct other types of analyses. For example, protein arrays can be used to: (1) screen for various molecules that interact with a protein of interest (e.g., agonists or antagonists). Molecules to be screened can include, but are not limited to, other proteins, nucleic acids, drugs, macromolecules or small molecules (see, e.g., Kramer, A., et al., (1993) Pept. Res. 6:314-319); (2) to map antibody epitopes (see, e.g., Kramer, A., et al., (1994) Methods 6:388-395; Reineke, U., et al., Mol. Diversity 1:141-148; Schneider-Mergener, J., et al., (1996) “Peptide Libraries Bound to Continuous Cellulose Membranes: Tools to Study Molecular Recognition” in Combinatorial Libraries, (Cortese, R., ed.), W. de Gruyter, Berlin, pp. 53-68; and Kramer, A., et al., (1995) Mol. ImmunoL 32:459-465); and (3) determine the cellular location of proteins of interest. Each of these applications is discussed in additional detail below.

2. Immunoassays

One major utility of the semiconductor nanocrystals is to utilize them to label either the antigen or an antibody in various types of immunological assays. As noted generally above, the use of semiconductor nanocrystals can permit multicolor detection thereby allowing multiple assays to be conducted simultaneously. Unlike standard ELISA methods, there is no need to wait for an enzyme to generate a detectable signal. Further, the photostability of semiconductor nanocrystals provides for increased detection sensitivity relative to organic fluorophores because the resistance to photobleaching allows for longer signal acquisition.

The immunoassays can be conducted in a variety of different formats. In some methods, the ligands (potential antigens) to be screened are attached to an array and then contacted with antibodies that are labeled with semiconductor nanocrystals. Alternatively, antibodies can be positioned on a support and then contacted with samples containing ligands that are labeled with semiconductor nanocrystals. In a third format, the ligand being screened is an antibody and it is attached to a support and then screened with a known antigen of interest. Regardless of the particular approach, ligands or antibodies that do not form a binding complex on the array are typically washed from the array. Complexes on the array are then detected by detecting a signal from a semiconductor nanocrystal within a complex.

The assays can also be performed in a “sandwich” type format in which antibodies positioned on an array are contacted with a sample containing ligands. The ligands in the sample can be labeled or unlabeled. Ligands that specifically bind to an antibody form a binary antibody-ligand complex. Thus, the antibodies on the array act to capture a ligand to which it specifically binds; hence, such antibodies are sometimes called “capture antibodies.” The binary complex formed between a capture antibody and a ligand can optionally be detected if the ligands are labeled. The array is also contacted with a secondary antibody that is labeled with a semiconductor nanocrystal. In those instances in which a secondary antibody specifically binds to a binary complex, a tertiary complex is formed. Tertiary complexes can be detected by detecting an emission mediated by the semiconductor nanocrystal in the tertiary complex.

As a further example, with certain immunoassays the wells of a microtiter plate are coated with a selected antigen. A biological sample containing or suspected of containing antibodies to the antigen is then added to the coated wells. After a period of incubation sufficient to allow antibody binding to the immobilized antigen, the plate(s) can be washed to remove unbound antibodies and other sample components and a detection moiety labeled with a semiconductor nanocrystal is added. The detection moiety is allowed to react with any captured sample antibodies, the plate washed and the presence of the secondary binding molecule detected as described above.

Thus, in one particular embodiment, the presence of antibodies bound to antigens immobilized on a solid support can be readily detected using a detection moiety that comprises an antibody labeled with a semiconductor nanocrystal that specifically binds to the antigen/antibody complex.

In still other methods, an immunoaffinity matrix can be provided, wherein a polyclonal population of antibodies from a biological sample suspected of containing a particular antigen is immobilized to a substrate. In this regard, an initial affinity purification of the sample can be carried out using immobilized antigens. The resultant sample preparation thus only contains specific antibodies, avoiding potential nonspecific binding properties in the affinity support. A number of methods of immobilizing immunoglobulins (either intact or in specific fragments) at high yield and good retention of antigen binding activity are known in the art. Not being limited by any particular method, immobilized protein A or protein G can be used to immobilize immunoglobulins.

Accordingly, once the immunoglobulin molecules have been immobilized to provide an immunoaffinity matrix, semiconductor nanocrystal-labeled proteins (i.e., potential antigens) are contacted with the bound antibodies under suitable binding conditions. After any nonspecifically bound antigen has been washed from the immunoaffinity support, the presence of bound antigen can be determined by assaying for a signal mediated by a semiconductor nanocrystal.

Additionally, antibodies raised to particular antigens, rather than the antigens themselves, can be used in the above-described assays in order to detect the presence of a protein of interest in a given sample. These assays are performed essentially as described above and are well known to those of skill in the art.

The ligands or antibodies used to prepare the array can be placed on a variety of different supports. Suitable supports for use in the methods of the invention include, but are not limited to, nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads; and magnetically responsive beads.

Sometimes, immobilization to a support can be enhanced by first coupling the ligand or antibody to a protein with better solid phase-binding properties. Suitable coupling proteins include, but are not limited to, macromolecules such as serum albumins including bovine serum albumin (BSA), keyhole limpet hemocyanin, immunoglobulin molecules, thyroglobulin, ovalbumin, and other proteins well known to those skilled in the art. Other reagents that can be used to bind molecules to the support include polysaccharides, polylactic acids, polyglycolic acids, polymeric amino acids and amino acid copolymers. Additional details regarding molecules and coupling methods are provided by, for example, Brinkley, M. A. (1992) Bioconjugate Chem. 3:2-13; Hashida et al. (1984) J Appl. Biochem. 6:56-63; and Anjaneyulu and Staros (1987) International J. ofPeptide and Protein Res. 30:117-124.

3. Antibody Generation for Immunoassays

Antibodies that are used in the methods of the invention are produced using established techniques and disclosed in, for example, U.S. Pat. Nos. 4,011,308; 4,722,890; 4,016,043; 3,876,504; 3,770,380; and 4,372,745. For example, polyclonal antibodies are generated by immunizing a suitable animal, such as a mouse, rat, rabbit, sheep or goat, with an antigen of interest. In order to enhance immunogenicity, the antigen can be linked to a carrier prior to immunization. Such carriers are well known to those of ordinary skill in the art.

Immunization is generally performed by mixing or emulsifying the antigen in saline, preferably in an adjuvant such as Freund's complete adjuvant, and injecting the mixture or emulsion parenterally (generally subcutaneously or intramuscularly). The animal is generally boosted 2-6 weeks later with one or more injections of the antigen in saline, preferably using Freund's incomplete adjuvant. Antibodies can also be generated by in vitro immunization, using methods known in the art. Polyclonal antiserum is then obtained from the immunized animal.

Monoclonal antibodies can be prepared using the method of Kohler and Milstein (1975) Nature 256:495-497, or a modification thereof. Typically, a mouse or rat is immunized as described above. However, rather than bleeding the animal to extract serum, the spleen (and optionally several large lymph nodes) is removed and dissociated into single cells. If desired, the spleen cells can be screened (after removal of nonspecifically adherent cells) by applying a cell suspension to a plate or well coated with the antigen. B-cells, expressing membrane-bound immunoglobulin specific for the antigen, will bind to the plate, and are not rinsed away with the rest of the suspension. Resulting B-cells, or all dissociated spleen cells, are then induced to fuse with myeloma cells to form hybridomas, and are cultured in a selective medium (e.g., hypoxanthine, aminopterin, thymidine medium, “HAT”). The resulting hybridomas are plated by limiting dilution, and are assayed for the production of antibodies which bind specifically to the immunizing antigen (and which do not bind to unrelated antigens). The selected monoclonal antibody-secreting hybridomas are then cultured either in vitro (e.g., in tissue culture bottles or hollow fiber reactors), or in vivo (e.g., as ascites in mice).

Human monoclonal antibodies are obtained by using human rather than murine hybridomas. See, e.g., Cote, et al. (1985) Monoclonal Antibodies and Cancer Therapy, Alan R. Liss, p. 77.

Monoclonal antibodies or portions thereof can be identified by first screening a B-cell cDNA library for DNA molecules that encode antibodies that specifically bind to p185, according to the method generally set forth by Huse et al. (1989) Science 246:1275-1281. The DNA molecule can then be cloned and amplified to obtain sequences that encode the antibody (or binding domain) of the desired specificity.

As indicated supra, a variety of other types of antibodies can also be utilized. For example, the antibodies can be recombinant antibodies from phage libraries. A variety of antibody fragments can also be used. Such fragments include Fab fragments, scFv fragments and mini-antibodies, for example.

Once formed, the antibodies can be labeled with semiconductor nanocrystals using the conjugation methods set forth infra.

D. Exemplary Assays

1. Whole Genome Screening

Certain techniques can be utilized to prepare large numbers of fusion proteins that subsequently can be screened for their ability to interact with macromolecules such as other proteins, nucleic acids, small molecules, oligosaccharides, drugs and other biological molecules. As noted supra, an array of cells expressing different fusion proteins comprising different ORFs from various organisms (e.g., yeast) can be constructed (see, e.g., Martzen, M. R., et al., (1999) Science 286:1153-1155). The proteins expressed by the cells can then be screened with ligands of interest to identify those capable of interacting with the expressed proteins. Such methods allow one to identify unknown ligands to known proteins, as well permitting one to identify unknown proteins (i.e., no activity has yet been assigned to the ORF) capable of binding known or unknown ligands.

2. Cellular Localization of Proteins

One can also conduct experiments to identify the location of various proteins throughout a cell using semiconductor nanocrystal labeled reagents. For example, recently developed transposon tagging schemes can be utilized. In this approach, transposon constructs that include a transposon flanked by recombination sites are prepared. The construct also includes an epitope tag segment adjacent one of the recombination sites. By the process of homologous recombination, a transposon construct can become integrated into the genome of the organism (e.g., yeast) being transformed. When the construct is inserted in frame with a coding region, a full-length epitope tagged protein is generated.

An array of such cells can then be assayed to determine the subcellular localization of various proteins. Since in frame insertions of the construct generates fusion proteins that include the epitope, the fusion proteins can be localized by contacting cells with antibodies that specifically bind to the epitope. In this way, one can localize proteins to various regions of the cell, such as the nucleus, mitochondria and plasma membrane, for example. See, e.g., Ross-Macdonald, P., et al. (1999) Nature 402:413-418. Detection of complexes formed between the antibodies and the localized proteins is enhanced by using antibodies that are labeled with semiconductor nanocrystals.

3. Epitope Determination

Semiconductor labeled antibodies can also be utilized to determine the antigenic epitope of antigens of interest. An antigenic epitope is defined as a region of a protein to which an antibody can bind. An immunogenic epitope refers to those parts of a protein that elicit the antibody response when the whole protein is the immunogen. Certain of these methods involve the synthesis of overlapping proteins that cover the entire amino acid sequence of protein known to elicit an antigenic response. These proteins can be synthesized in an array format using solid state protein synthesis methods such as described supra. The immobilized proteins are then tested for antigenicity using established ELISA techniques and various different anti-sera in which the antibodies are labeled with semiconductor nanocrystals. Once a peptide is identified that forms a stable complex with an antibody generated against the natural antigen, a replacement set of proteins is generated. The replacement set includes all the proteins corresponding to the identified protein except that a single amino acid replacement is introduced. Each replacement set also can be synthesized as an array. The replacement set is then rescreened using antibodies labeled with semiconductor nanocrystals to determine if antigenicity is retained. From the collective results, one can determine the location and identity of amino acids that play a critical role in antigenicity. See, e.g., Geysen, H. M., et al. (1984) Proc. Natl. Acad. Sci. USA 81:3998-4002.

VII. Tissue Arrays

A. Background

Histopathological examination in which tissue specimens are subjected to microscopic examination has enabled the biological mechanisms of many diseases to be clarified and consequently aided in the development of effective medical treatment for a variety of illnesses. In traditional pathological analysis, a diagnosis is made on the basis of cell morphology and staining characteristics. Such analyses can sometimes be limited because of the sensitivity of standard staining procedures.

Developments in the field of molecular medicine have generated new methods for investigating the cellular mechanisms of disease and to determine the most appropriate treatment course. For example, often certain diseases are associated with an alteration (i.e., decrease or elevation) in the expression level of certain proteins. As a specific example, it has been found that certain hormone dependent breast tumor cells have increased expression of estrogen receptors. Other diseases have been correlated with the production of cell surface antigens or the generation of other cellular proteins. Various detection techniques have been developed to identify and detect such markers, including, for example, immunophenotyping with monoclonal antibodies and in situ hybridization with probes specific for the nucleic acid sequences encoding such markers. To improve the throughput at which samples can be analyzed, tissue arrays can be utilized to permit the rapid screening of a large number of tissue samples.

Semiconductor nanocrystals can be used in all of the foregoing methods to enhance detection, sensitivity and, in some instances, to provide quantitative information. The semiconductor nanocrystals can be conjugated to a number of different species to stain particular cellular components. For example, as described above, semiconductor nanocrystals can be conjugated to nucleic acids and antibodies and then used to probe the presence or absence of particular proteins or nucleic acids within a tissue specimen.

B. Tissue Array Preparation

Tissue arrays can be prepared in a number of different ways. A clinician can obtain tissue samples from various sources using standard anatomical procedures and individually place the tissue samples at different locations on some type of a support (e.g., a glass slide or microtiter wells). The process can be automated in certain respects using devices designed to obtain tissue samples and then place the samples within an array. An example of suitable devices for preparing arrays in this manner are discussed in PCT publications WO 00/24940, 99/44062 and 99/44063, which are incorporated herein by reference in their entirety. In general these devices utilize a punch apparatus to bore into a tissue sample and then dislodge the sample into a receptacle in an array support.

The array can be prepared in a variety of different formats. For example, the array can include tissue samples from a single individual. Different labeled biomolecules bearing a semiconductor nanocrystal can be added to each location to detect the presence of a different cellular species. Other arrays can include tissue samples from a number of different individuals. For instance, the array can include tissue samples obtained from the same type of tissue from different individuals to screen for particular disease markers. A specific example of such an array is an array of breast tissue samples taken from women suspected of having breast cancer. Sets of tissue arrays can also be prepared, each set of arrays including the same tissue samples. Each array can be subjected to a different type of analysis to detect different markers. In this way, results for a number of different markers can rapidly be compiled for a population of different individuals. As discussed further below, however, the multicolor capabilities of semiconductor nanocrystals permits a number of different types of interrogations to be conducted with a single tissue sample. Thus, utilization of the multiplexing capabilities of semiconductor nanocrystals coupled with an array format in which sets of arrays having the same tissue samples on each array allows for very high throughput analysis of a number of different markers.

Various types of tissue samples can be utilized. Suitable tissue samples include, but are not limited to, tissue sections excised using known surgical procedures, one or more whole cells, cellular suspensions, cytological preparations (e.g., smears obtained from cellular suspensions), as well as cellular extracts or homogenates. Cell suspensions can be utilized, for example, by pelleting the suspension and then fixing it to a support. The samples can also be obtained from specific tissues (e.g., skin, breast, prostrate, testes and ovaries) or from various bodily fluids (e.g., blood, plasma, urine that contains cells, semen, vaginal fluids, bronchial washings and ascitic fluids). In the case or cell suspensions or extracts, the cells can be directly obtained from an organism or can be obtained from a cell culture.

C. Assays

Tissue arrays can be used to rapidly profile hundreds or thousands of tissue samples at the DNA, RNA and protein levels. However, the assays are not limited to detecting these three major classes of biomolecules. The presence and concentration of oligosaccharides, glycoproteins, particular fatty acids and other targets can all be detected using an appropriate molecule that specifically binds to the target of interest Because semiconductor nanocrystals can be tuned to a variety of different wavelengths, it is possible to use semiconductor nanocrystal labeled nucleic acids, proteins (e.g., antibodies) and other biomolecules to probe multiple different DNAs, RNAs, proteins and/or other biomolecules within a single tissue sample. The results from such investigations can be compiled in a database.

The use of semiconductor nanocrystal-conjugates (either a single semiconductor nanocrystal conjugated to biomolecules or a plurality of semiconductor nanocrystals) allow specific, sensitive, photostable detection of target molecules, factors that can be problematic using currently available stains. Additionally the inherent properties of semiconductor nanocrystals, i.e., single excitation source, narrow, gaussian spectra and tunability of emission wavelength, mean that many more colors are resolved than with conventional fluorescent dyes.

Various formats can be used to conduct analyses utilizing tissue arrays. As indicated above, samples can be interrogated to detect the levels of target nucleic acids using either semiconductor labeled nucleic acids whose sequence is complementary to the target nucleic acid being interrogated or a labeled protein that specially binds to the target nucleic acid. When the level of a protein is to be monitored, typically an antibody that specifically binds to the target protein and bears a semiconductor nanocrystal is used to detect the presence of the target protein. However, if the target protein is a DNA binding protein, then the nucleic acid that binds to the protein can be labeled with a semiconductor nanocrystal and used to probe for the target protein.

When labeled antibodies are utilized, analyses can be carried out in a two-step reaction in which a primary antibody is initially contacted with the tissue sample, followed by addition of a semiconductor nanocrystal-conjugated antibody. Alternatively, an antibody (or other biomolecule) semiconductor nanocrystal conjugate can be used to directly label proteins of interest within the sample. As a specific example, five (or more with increased spectral use or reduced spectral separation) different populations of semiconductor nanocrystals can be synthesized with emission spectra that are spaced at 40 nm intervals from, e.g., 490-650 nm. Each spectrally distinct population of semiconductor nanocrystals is conjugated to a different molecule that specifically recognizes a biomolecule of interest that may or may not be present in the sample to be analyzed. Following standard protocols, the sample is labeled with the semiconductor nanocrystals and analyzed for the location and quantity of the target molecule. This analysis may be carried out by conventional fluorescent microscopy techniques or by use of a spectral scanning device.

Since many semiconductor nanocrystals can be generated that are spectrally distinct, it is possible to label different biomolecules such as multiple antibodies and/or multiple nucleic acid probes that can then be used to measure the position and quantity of cellular compounds. When different compounds are not colocalized in the cell, many more semiconductor nanocrystal colors can be used by taking advantage of the known spatial separation of the targets to be analyzed. For example, no overlap would occur between nuclear localized targets and membrane localized targets. Hence organelle- specific groups of semiconductor nanocrystals can be employed to increase dramatically the number of discemable targets.

More specifically, the multi-color capability of semiconductor nanocrystals can be used, for example, to conduct multi-color in-situ hybridization to measure the levels of tRNA, MRNA, DNA, protein or any other cellular compound that can be stained in a tissue array. By using multiple colors on one array rather than using multiple arrays with single color detection, greater accuracy is achieved, since the tissue sections are not homogeneous and can therefore vary slightly from array to array. This variance can easily effect assay results. Utilization of multi-color detection with semiconductor nanocrystals reduces this problem. In addition, as described in greater detail below, by utilizing semiconductor nanocrystal conjugates that include a single semiconductor nanocrystal, one can make quantitative measurements of the target biomolecule(s) under investigation. Multiplexing also allows rapid analysis of many molecular markers in the same set of specimens with greater accuracy.

The use of semiconductor nanocrystal labeled biomolecules in assays of tissue arrays can be utilized in a variety of applications. One application is to use tissue arrays in various types of correlation studies. For instance, by obtaining samples from individuals known to have a particular disease, analyses with various semiconductor nanocrystal conjugates can be used to identify potential markers associated with the disease or illness. In general, such methods involve the use of semiconductor nanocrystal conjugates to determine the levels of selected DNA, RNA and/or protein levels in tissues from diseased individuals. Detection of elevated or reduced levels of various genes or gene products can be used to make initial correlations between such genes or proteins with the particular disease under investigation.

In a related fashion, tissue array assays utilizing semiconductor nanocrystal conjugates can be used to establish correlations between certain markers and patient prognosis. The levels of various DNA, RNA and/or protein levels can be monitored over time in tissue samples obtained from patients known to have a disease as the patients receive different treatments. By monitoring the health history of the patients and the various levels of markers over time, one can establish correlations between certain markers and disease outcomes. Similarly, correlations between levels of markers and the efficacy of different therapeutic treatments can established. In this instance, the level of different markers is tracked for individuals receiving different treatments. The photostability of semiconductor nanocrystals allows tissue arrays t be read repeatedly and archived for the purpose of comparison at a later date.

Once a correlation has been established between a disease and one or more markers, semiconductor nanocrystal conjugates that specially recognize such markers can be used to screen and identify individuals that have a disease or are susceptible to acquiring the disease. In like manner, once correlations between marker profiles and treatment efficacy have been established, assays utilizing semiconductor nanocrystal conjugates can be utilized to determine the marker profile for an individual and thus identify the most appropriate treatment option.

Tissue array analysis can also be used in combination with other analyses. For example, the initial correlation studies just described can be conducted using the differential gene expression methods described supra. Hence, nucleic acid arrays can be utilized to identify a nucleic acid whose expression is altered in diseased individuals. Additional validation studies can then be conducted using tissue arrays. If the nucleic acid is shown to be a bonafide marker for a disease, then semiconductor nanocrystal conjugates that specifically bind to the marker can be used to screen individuals for the presence of, or susceptibility to, the disease.

VIII. Secondary Interrogations

Semiconductor nanocrystals can be utilized to label various target biomolecules for use in various types of secondary interrogations. Such investigations generally involve conducting an additional analysis once a binding a binding complex between two or more biomolecules have already been formed. The array in such investigations typically bears a biomolecule that captures a target molecule in preparation for a secondary interrogation. Suitable targets in this type of study include, but are not limited to, nucleic acids (e.g., DNA, RNA), proteins, or antibodies.

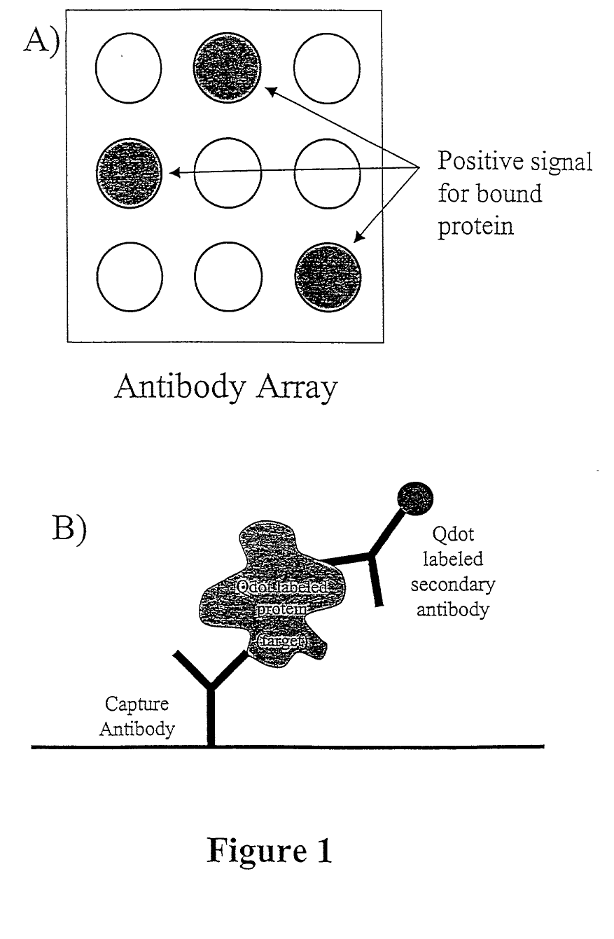

A specific example of a secondary interrogation is the use of an array of antibodies to probe for multiple epitopes of a protein. In this instance, each spot on the array contains a different antibody. A complex protein target is labeled with a single color semiconductor nanocrystal and allowed to bind to the array bearing the different antibodies. In the case of FIG. 1A, the three shaded regions correspond to the positive signals from the array. Once the target molecules have been bound to the array, a secondary antibody (labeled with a semiconductor nanocrystal having a second color) is brought into contact with the array and given the opportunity to bind to a second epitope of the bound proteins (FIG. 1B). In the specific example shown in FIG. 1A, different colors are used to label each of the three active antibodies identified in the first round of screening.

This procedure can be used, for example, to find complementary antibody pairs. In the first step, several antibodies are identified that bind efficiently to the target molecule. Since each of these antibodies may bind to a different epitope of the target molecule, the second step can interrogate the bound proteins by contacting the array with the same antibodies identified in the first step. Those locations on the array that emit signals from both semiconductor nanocrystals are those locations in which there are two antibodies that recognize different epitopes of the target molecule.

This technique of capture and secondary interrogation can also be used to do simultaneous genotyping of multiple SNPs. If 2 SNPs are located far apart on the target nucleic acid under analysis, both polymorphic sites can be interrogated simultaneously by using a nucleic acid probe on an array that is complimentary to the first SNP to capture the target nucleic acid labeled with a semiconductor nanocrystal. Once the labeled target nucleic acid is captured, it can be probed with a second set of labeled probes that is complimentary to the various polymorphic forms of the second SNP. Probes for different polymorphic forms can each be labeled with a different color. By contacting the array with the second set of probes, each SNP can be identified by color and position, and the haplotype can be determined.

IX. Expansion of Dynamic Range and Single Copy Counting

A. General

As noted above, two key shortcomings associated with existing array methods concern limitations on sensitivity at low concentrations and limitations on dynamic range, namely the ability to accurately and simultaneously measure target concentration over a wide range of concentrations. With the fluorescent labels currently utilized to conduct array analyses, one often is forced to sacrifice linearity at high concentrations for detection sensitivity at low concentrations.

The use of semiconductor nanocrystals can provide significant improvement in both sensitivity and dynamic range. The fluorescence from semiconductor nanocrystals is extremely bright and stable, and permits routine detection of single semiconductor nanocrystals (see, e.g., Empedocles et al. (1999a), “Threedimensional orientation measurements of symmetric single chromophores using polarization microscopy,” Nature 399:126-130; Empedocles et al. (1999b), “Spectroscopy of Single CdSe Nanocrystallites,” Acc. Chem. Res. 32:389-396; Empedocles et al. (1997), “Quantum-confined Stark effect in single CdSe nanocrystallite quantum dots,” Science 278:2114-2117; Empedocles et al. (1996), “Photoluminescence spectroscopy of Single CdSe nanocrystallite quantum dots,” Phys. Rev. Lett. 77:3873-3876). The ability to detect single labels dramatically increases detection sensitivity. This feature of semiconductor nanocrystals means detection can be extended into the single copy counting regime. The increase in sensitivity afforded by semiconductor nanocrystals enables the detection of minute quantities of target molecule. This is important in a variety of assays in which the target is present at only very low concentrations, such as detecting subtle changes in gene expression (changes caused, for example, by disease or environmental changes).

Even when dealing with target molecule concentrations that can be detected using current fluorophores, there are still problems associated with the quantitative analysis of array results using organic dyes. Inconsistent performance of dyes such as Cy5 can result in unreliable array results. Semiconductor nanocrystals can act as a more stable and reliable substitute for existing fluorophores such as Cy3 and Cy5. In addition, typically semiconductor nanocrystals do not self quench to the extent that organic dyes do, remaining capable of producing strong luminescence even when they are packed into solid semiconductor nanocrystal films. This means that in some instances semiconductor nanocrystals can also expand the high-end limit of concentration detection over what can be achieved using standard fluorophores. Hence, the characteristics of semiconductor nanocrystals allows detection to occur at lower and higher concentration levels, thus expanding the dynamic range of detection.

The single target counting assays and methods thereof described herein do not necessarily have to be performed using semiconductor nanocrystal labels. Any fluorescent label capable of being detected on the single molecule level can be utilized for the type of measurement described herein. Hence, suitable labels include, but is not limited to, organic dye molecules, metal colloid scattering particles, and surface-enhanced Raman spectroscopy (SERS) particles. However, the many unique features of semiconductor nanocrystals described supra, make them particularly useful as labels in single target counting assays. In addition, certain methods described herein do not require the ability to detect a single label, but rather a single target molecule. Therefore, the methods described herein can be used to detect single target molecules that are labeled with a single detectable label, or with multiple detectable labels.

B. Basis for Single Copy Counting and Dynamic Range Expansion

The high stability, detection sensitivity and ease of multiplexing make semiconductor nanocrystals useful as multi-color fluorophores for use in ultra-sensitive surface based assays. The ability to easily detect single semiconductor nanocrystals means that semiconductor nanocrystals are particularly useful as fluorophores in bioassays in which single target molecules bound to the assay surface are counted one at a time.

“Single target counting” does not mean the counting of all of the target molecules within a sample, but rather the counting of the target molecules that are bound to the surface of the array substrate. While this number is not necessarily the same as the total number of target molecules in the sample, the actual target level can be determined through calibration against a sample of known concentration. By enabling the detection and counting of single bound target molecules, one can extend the sensitivity of surface-based assays beyond what is possible using current detection techniques. For instance, current microarray technology allows the detection of target at a density of as low as 0.1 labels/μm² (˜8 labels per 10 μm diameter confocal spot). With single target counting, the theoretical limit of detection is 1 label per array spot, extending the detection sensitivity by as much as 3 orders of magnitude for a 100 μm diameter array element.

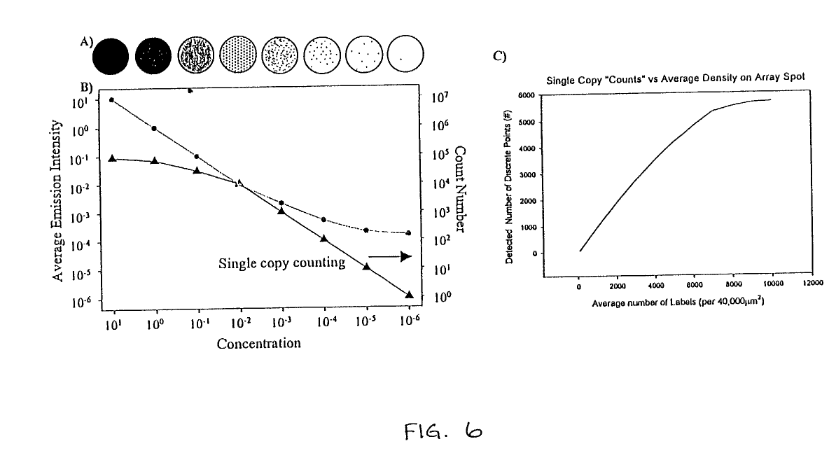

In order to properly understand how the detection of single bound target molecules improves the sensitivity and dynamic range of a surface-based assay, it is important to understand what is actually measured at the high and low end of the concentration range on an assay surface. For the purposes of illustration, a microarray will be considered; however, the ideas presented for the microarray also hold true for any surface-based assay. FIG. 6A shows a graphic representation of a series of microarray spots with decreasing concentrations of bound target. The left side corresponds to the high concentration regime (ensemble regime), in which the entire array spot is covered with target and the average emission intensity is dependent on the average density of label across the surface of the array. In this regime, sample concentration is proportional to average emission intensity (ensemble intensity). The right side corresponds to the single copy counting regime, where individual bound target molecules are separated from each other by distances that are greater than the diffraction limit of light and can be detected one at a time. In this regime, sample concentration is proportional to the number of individual targets counted on the surface of the array.

FIG. 6B shows data simulating the relative signal vs concentration detected using ensemble intensity and single copy counting over the entire concentration range. Ensemble measurements yield a linear concentration dependence at high concentrations, but saturate at low concentrations. This saturation occurs when the total signal from bound target in the detection region is lower than the noise generated from the integrated background across that entire region. Detecting single molecules bound to the array with high-resolution microscopy, however, can dramatically reduce the integrated background noise by comparing the signal from a single fluorophore to the background from an extremely small (diffraction limited) area of the array spot.

As an example, if the background signal increases linearly with total detection area, then the background generated over a standard 10 μm diameter ensemble probe spot is 400 times higher than the background generated from a high resolution image of a single fluorophore (˜0.5 μm diameter). This results in a decrease in noise (and therefore an increase in sensitivity) of a factor of 20. This effect is further enhanced if the ensemble signal is integrated over the entire array spot. For a 100 μm diameter spot, the background signal is 40000 times higher than for a diffraction limited spot resulting in approximately 200 times higher sensitivity. The background over the bottom of an entire well of a 96 well plate is ˜10⁸ times higher yielding an enhancement of 10⁴. To achieve these enhancements, however, one needs to be able to detect the fluorescence from a single bound target molecule with high spatial resolution.

In contrast to ensemble intensity measurements, the single target counting signal saturates at high concentrations. This occurs when the concentration increases to the point where individual target molecules are so close together that they can not be distinguished. This means that some individual spots actually contain more than one bound target molecule and therefore results in an undercounting of the total number of target molecules. The result is an underestimate of the total sample concentration (see FIG. 6C).

Between the ensemble and single target counting regime, there is a regime in which the concentration is low enough to count individual targets, but high enough to be detectable in an ensemble measurement. This is referred to as the transition regime. Preliminary measurements of single semiconductor nanocrystals nonspecifically adsorbed to a surface over a wide range of concentrations suggests that it should be possible to calibrate the transition regime using either ensemble or single target counting, allowing the user to calibrate concentrations across all regimes.

By combining single copy counting and ensemble intensity measurements, detection sensitivity can be increased, as well as the dynamic range of microarray assays. In standard measurements, detection sensitivity at the low end is achieved at the expense of dynamic range at the high end due to detector saturation. However, by combining single target counting with ensemble intensity measurements, one can cover the entire dynamic range in a single experiment. The reason is that in the single copy counting regime, as the concentration increases, the peak intensity does not—only the number of detected spots increases. As such the entire dynamic range of the detector can be used to cover the ensemble concentration regime, where peak intensity varies linearly with concentration.

As an example, consider a detection system capable of detecting the fluorescence from single semiconductor nanocrystals over the entire area of a 100 μm microarray spot, with a spatial resolution of less than 0.5 μm. This system uses a 2dimensional CCD camera with a dynamic range of 65,536 counts per pixel and a read noise of ˜2 counts/pixel. If excitation intensity and integration time are selected to yield 30 counts/pixel/semiconductor nanocrystal, then in the single copy counting regime, individual semiconductor nanocrystals are detected with a signal to noise ratio of ˜15. Assuming an even distribution of bound molecules and a spatial resolution of 0.5 μm, at best one can detect 40,000 individual spots within each 100 μm array spot. In an ideal system, this would result in a dynamic range within the single target counting regime of more than 10⁴. As the concentration increases into the ensemble regime, the average intensity increases linearly with concentration. The detector then provides an additional dynamic range of 10³ before saturating. As a result, a total dynamic range of 10⁷ is theoretically possible in a single experiment. Of course, multiple integration times can be used to extend the dynamic range to higher concentrations if necessary.

C. Detection

Single molecule fluorescence detection can be achieved using either laser scanning confocal microscopy or wide-field imaging with a 2D CCD camera. One distinct advantage of wide-field imaging over scanning confocal microscopy for these applications is that fluorescence can be collected from all points within one or more array spots simultaneously. This means that the signal can be integrated for relatively long periods of time without increasing the read-time for the array. This is particularly beneficial when detecting semiconductor nanocrystals, since they do not photobleach. It is therefore possible to integrate the signal from each array spot for a relatively long time compared to organic dyes. This can provide significantly higher fluorescence signal and is one of the things that allows one to easily detect the fluorescence from single semiconductor nanocrystals. At the same time, however, a relatively high spatial resolution (<0.5 μm) is needed in order to be able to spatially separate the fluorescence from individual target molecules and maximize the signal to background ratio. The combination of high spatial resolution with long integration times can be prohibitive when using a confocal scanning optical system. For instance, using a confocal scanning system with a 0.5 μm resolution and an integration time of 100 ms would take more than 15 minutes to scan a single 100 μm diameter microarray spot. This is a prohibitively long time when considering arrays with 10000 spots (100 days).

Using wide-field detection and a high numerical aperture microscope objective, the same image of a single spot can be obtained in a single 100 ms exposure. Once taken, the array can be translated to an adjacent region and the next image acquired. By precisely controlling the scanning stage and stitching the images together, the entire array image can be produced. This procedure dramatically decreases the total read-time, allowing an entire array to be read in less than 20 minutes. In addition, in some instances multiple array spots can be imaged simultaneously, further reducing the total collection time.

While the methods of the present invention focus on array based assays in which an assay occurs on an array surface, the methods also apply to other bioassays performed on a surface support such as the bottom of a microtitre plate or a polymer bead. The considerations just described apply generally to any of the assays set forth above. Assays typically are conducted with semiconductor nanocrystals, but as noted above, can also be performed with other fluorophores such as an organic dye or metal colloid. Semiconductor nanocrystals can be incorporated into the ligands or antiligands of the assay via a plurality of techniques described herein. Each bound target molecule is labeled with 1 or more semiconductor nanocrystals.

Once the assay is complete and one or more complexes containing semiconductor nanocrystals have been formed, the fluorescence from the sample is detected. If the density of bound target molecules is greater than ˜1 target/μm², then the assay signal is measured and calibrated using the total emission intensity from the entire assay region (e.g. the total signal from a single microarray spot or from an entire microtitre well). If the target density is less than ˜1 target/μm², so that individual target molecules can be spatially resolved using standard far-field optics, then the assay signal is measured and calibrated by counting the total number of bound target molecules. The assay signal can be measured from all assays using both ensemble and single target counting methods. A calibration curve can then be used to identify which assays fall in the ensemble, single and transition regimes.

Typically, complexes including semiconductor nanocrystals are detected with an optical detection system capable of detecting the fluorescence from single semiconductor nanocrystals (or other labels) with a spatial resolution of 1 μm or less. In some instances, this optical system is comprised of a wide-field imaging system with a 2D CCD camera and a high numerical aperture microscope objective. A laser based microscope system capable of detecting and spectrally resolving the fluorescence from single semiconductor nanocrystals can also be utilized (see, e.g., Empedocles et al. (1999a), Nature 399:126-130; Empedocles et al. (1999b), Acc. Chem. Res. 32:389-396; Empedocles et al. (1997), Science 278:2114-2117; and Empedocles et al. (1996), Phys. Rev. Lett. 77:3873-3876).

The optical design of the laser based microscope system is based on a wide-field epifluorescence microscope. FIG. 7 is a schematic drawing of the significant optical components of such a laser microscope system 100. Excitation light 102 from a laser source (488 nm Ar+) 104 is transmitted through a dispersing prism 106 and a 500 nm short pass dichroic mirror 108 at an angle of 45°. The excitation light is then focused by a high numerical aperture microscope objective 110 onto the sample surface 112. An additional lens in the excitation path (i.e., the dispersing prism 106) causes the laser 104 to illuminate a wide area of the sample surface 112. The fluorescent image is collected by the same objective lens 110. The image is reflected by the dichroic mirror 108, passes through a wavelength specific filter 114 to remove any excitation light, and is focused by a final lens 116 onto the detection system 118. The detection system 118 consists of a 2D CCD camera 120 and a tunable bandpass filter 122. Spectral images are obtained by acquiring multiple images at a different wavelength. With this system, it is possible to simultaneously obtain spectra at every point within the image with a spectral resolution of 2 nm and a spatial resolution of less than ˜0.5 μm. Uniform excitation intensity in this system can be generated either through the use of a lamp light source or a laser excitation source that has been transformed from a Gaussian intensity profile to a “top-hat” profile through the use of a series of 2 Powel lenses each oriented at 90 degrees relative to each other. Alternatively, the optical system can be comprised of a scanning confocal microscope system with a spatial resolution of less than ˜0.5 μm.

Another detection option utilizes an optical system that comprises a microscope with an immersion microscope objective in which the sample is viewed from the backside of the sample substrate (e.g. from the underside of a microarray substrate or from the bottom of a microtitre well). In certain instances, the sample can be located on the surface of a glass or quartz substrate and is detected with a high numerical aperture oil-immersion microscope and index matching immersion oil (n=1.51). This can yield an increase in collection efficiency of as much as 800%. Alternatively, detection can be with a water- or other fluid-immersion lens, also detecting from the back-side of the sample substrate.

For ultrasensitive detection of single target molecules, it is not only necessary to have a bright fluorophore, but also to minimize the collection of background fluorescence from the substrate surface and assay materials. Autofluorescence from the array substrate and assay materials can be minimized by (a) using low fluorescence array substrates such as quartz or low fluorescence glass, (b) choosing a fluorescent label that does not overlap significantly with the autofluorescence from the substrate and assay materials, and (c) choosing an excitation wavelength that does not significantly excite autofluorescence. Since semiconductor nanocrystals can be synthesized to absorb and emit at any wavelengths, they are an ideal fluorophore for minimizing interference from autofluorescence.

An important issue in detecting assays on the single target counting level is how to locate assay regions with very low signal. For instance, if a microarray is labeled at a density in the single target counting regime, in some instance it can be difficult to locate the array spots for quantitative detection. In certain methods, kinematic alignment of the array slide combined with the use of “alignment spots” is used to automatically locate the edges of the array and register the first image so that the array spots are each located within the center of each image. Alignment spots are array spots that are not complementary to any sequence of interest. To each hybridization sample, one can add a labeled target that is specific for these alignment spots at a known concentration. These spots therefore have a high signal and can be detected and used for alignment purposes. A pattern of alignment spots can be placed across each array that unambiguously identify the absolute position of the array.

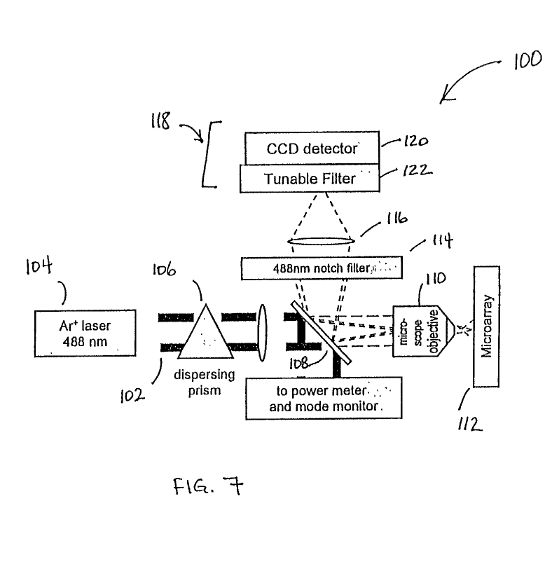

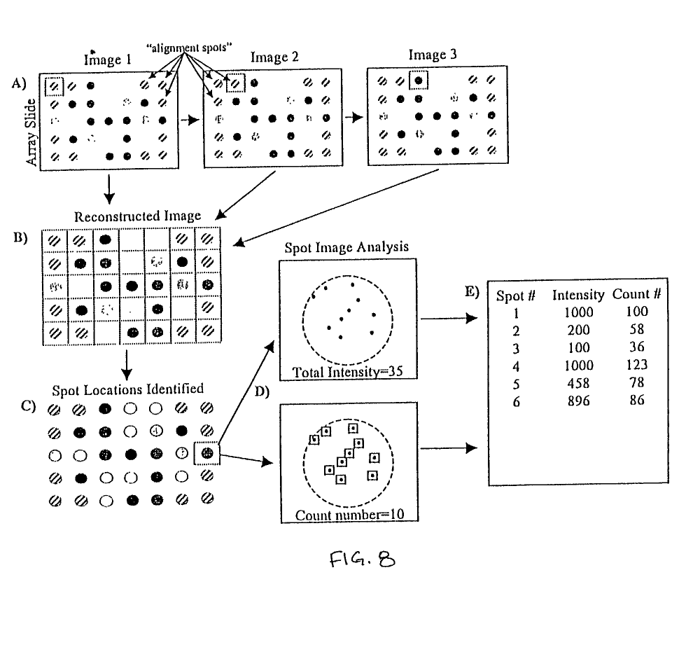

Software can then be used to locate and analyze each spot within the array. Using pattern recognition algorithms, the alignment spots can be identified and all other spot locations will be determined from the known periodicity of the array. Once the array pattern is determined, each spot on the array can be located according to its position within a periodic lattice. The radius of all spots is the same and can be predetermined or extracted from the radius of the alignment spots. Two separate algorithms can then be used to analyze the signal from within each spot area. First, the total integrated signal from within each spot can be measured and compared to either an equivalent area outside of the array spot or to a calibration spot of known intensity. Second, an algorithm can be used to count individual fluorescent point within each array spot. Using pattern recognition, the algorithm can identify and count fluorescent points that fit a set of predetermined characteristics of shape, size and threshold intensity that are specific for the fluorescence from single semiconductor nanocrystals. A data file can be exported containing the ensemble intensity and the “count number” (i.e. the number of discrete fluorescent points) for each spot. FIGS. 8A-8E describe the complete array scanning procedure.

For some surface based assays such as microtitre plate assays, macroscopic alignment of the optical system can be used (i.e. scanning the entire bottom of each microtitre well). For bead-based assays, it is possible to use a second semiconductor nanocrystal color that does not spectrally overlap with the detection label. This second color can be added to each bead, either internally, or bound to the surface at a known concentration. This color can then be used to locate individual beads. Once found, a bandpass filter can be used to block the fluorescence from the alignment color and allow single target detection of only the label semiconductor nanocrystals. This 2-color technique can also be used for microarrays.

One additional requirement for a successful assay system capable of detecting single bound target molecules is the minimization of nonspecific binding of the detection label. Too many nonspecifically bound semiconductor nanocrystals can interfere with the quantitative measure of target concentrations on the level of single target counting. Consequently, labeling of these assays typically is with a fluorophore with extremely low nonspecific binding. Because the surface of semiconductor nanocrystals can be modified to have virtually any functionality, one can optimize the surface characteristics of the semiconductor nanocrystals to minimize nonspecific binding.

In other methods, each target molecule is labeled with two different semiconductor nanocrystal colors via two different binding interactions. Specifically bound labels can then be identified through the detection of both colors colocalized within the same fluorescent spot.

D. Nonspecific Binding Identified by Single Color Fluorescence

The primary shortcoming of surface based assays such as nucleic acid microarrays is the lack of appropriate sensitivity needed to detect extremely low levels of target concentration. For instance, as much as 40% of the known genes of interest studied using gene expression microarrays are expressed at a level of between 1 and 10 copies per cell, just at or below the limit of detection using current detection schemes. In addition to low expression levels, the costs incurred in extracting material for genetic testing creates pressure to minimize sample size requirements for genetic analysis. The ability to measure vanishingly small quantities of expressed DNA significantly improves one's ability to identify and treat diseases at an early stage. Ultra-sensitive detection in microarray assays can also assist in identifying new genes of interest in all areas of disease. A system for labeling and high sensitivity detection of fluorescence from DNA microarrays can significantly reduce the costs associated with expression analysis while simultaneously increasing the available information content.

Currently, the preferred method for detection of surface based assays such as microarrays is by labeling target molecules with organic dyes. For DNA microarrays using organic dyes, the current state-of-the-art detection can only detect a minimum of approximately 10 molecules in a 10 μm×10 μm region of a microarray spot (Duggan et al. (1999) Nature Genetics 21(nls):10-14). This means that the minimum number of bound DNA molecules required in order to detect signal from a standard 100 μm diameter microarray spot is approximately 1000. In order to generate this signal, more than 10 million cells may be required. In many instances, it is not possible to extract this much cellular material. Using single target counting with semiconductor nanocrystals, the theoretical limit of detection is 1 molecule per array spot, reducing the amount of cellular material required by three orders of magnitude and significantly improving our ability to identify and monitor important expression profiles. Similarly, the ability to detect single bound target molecules in all types of bioassays will dramatically improve the sensitivity and dynamic range of these measurements, enhancing the information content and minimizing costs.

X. Preparation of Semiconductor Nanocrystals

Semiconductor nanocrystals for use in the subject methods are made using techniques known in the art. See, e.g., U.S. Pat. Nos. 6,048,616; 5,990,479; 5,690,807; 5,505,928; 5,262,357 (all of which are incorporated herein in their entireties); as well as PCT Publication No. 99/26299 (published May 27, 1999), which is also incorporated by reference in its entirety. In particular, exemplary materials for use as semiconductor nanocrystals in the biological and chemical assays of the present invention include, but are not limited to those described above, including group II-VI, III-V and group IV semiconductors such as ZnS, ZnSe, ZnTe, CdS, CdSe, CdTe, MgS, MgSe, MgTe, CaS, CaSe, CaTe, SrS, SrSe, SrTe, BaS, BaSe, BaTe, GaN, GaP, GaAs, GaSb, InP, InAs, InSb, AlS, AlP, AlSb, PbS, PbSe, Ge and Si and ternary and quaternary mixtures thereof. The semiconductor nanocrystals are characterized by their uniform nanometer size.

As discussed above, the selection of the composition of the semiconductor nanocrystal, as well as the size of the semiconductor nanocrystal, affects the characteristic spectral emission wavelength of the semiconductor nanocrystal. Thus, as one of ordinary skill in the art will realize, a particular composition of a semiconductor nanocrystal as listed above will be selected based upon the spectral region being monitored. For example, semiconductor nanocrystals that emit energy in the visible range include, but are not limited to, CdS, CdSe, CdTe, ZnSe, ZnTe, GaP, and GaAs.

Semiconductor nanocrystals that emit energy in the near IR range include, but are not limited to, InP, InAs, InSb, PbS, and PbSe. Finally, semiconductor nanocrystals that emit energy in the blue to near-ultraviolet include, but are not limited to, ZnS and GaN.

For any particular composition selected for the semiconductor nanocrystals to be used in the inventive methods, it is possible to tune the emission to a desired wavelength by controlling the size of the particular composition of the semiconductor nanocrystal. In some instances, 5-20 discrete emissions (five to twenty different size populations or distributions distinguishable from one another), more preferably 10-15 discrete emissions, are obtained for any particular composition, although one of ordinary skill in the art will realize that fewer than five emissions and more than twenty emissions can be used depending on the monodispersity of the semiconductor nanocrystal particles. If high information density is required, and thus a greater number of distinct emissions, the nanocrystals are preferably substantially monodisperse within the size range given above.

As explained above, “monodisperse,” as that term is used herein, means a colloidal system in which the suspended particles have substantially identical size and shape. In certain high information density applications, monodisperse particles deviate less than 10% rms in diameter, and preferably less than 5%. Monodisperse semiconductor nanocrystals have been described in detail in Murray et al. (1993) J. Am. Chem. Soc. 115:8706, and in Murray, “Synthesis and Characterization of II-VI Quantum Dots and Their Assembly into 3-D Quantum Dot Superlattices,” (1995) Doctoral dissertation, Massachusetts Institute of Technology, which are hereby incorporated by reference in their entireties. The number of discrete emissions that can be distinctly observed for a given composition depends not only upon the monodispersity of the particles, but also on the deconvolution techniques employed. Semiconductor nanocrystals, unlike dye molecules, can be easily modeled as Gaussians and therefore are more easily and more accurately deconvoluted.

However, for some applications, high information density is not required and it is more economically attractive to use more polydisperse particles. Thus, for applications that do not require high information density, the linewidth of the emission can be in the range of 40-60 nm.

In certain methods, the surface of the semiconductor nanocrystal is modified to enhance the efficiency of the emissions, by adding an overcoating layer to the semiconductor nanocrystal. The overcoating layer is typically utilized because at the surface of the semiconductor nanocrystal, surface defects can result in traps for electrons or holes that degrade the electrical and optical properties of the semiconductor nanocrystal. An insulating layer at the surface of the semiconductor nanocrystal provides an atomically abrupt jump in the chemical potential at the interface that eliminates energy states that can serve as traps for the electrons and holes. This results in higher efficiency in the luminescent process.

Suitable materials for the overcoating layer include semiconductor materials having a higher bandgap energy than the semiconductor nanocrystal core. In addition to having a bandgap energy greater than the semiconductor nanocrystal core, suitable materials for the overcoating layer should have good conduction and valence band offset with respect to the core semiconductor nanocrystal. Thus, the conduction band is desirably higher and the valence band is desirably lower than those of the core semiconductor nanocrystal. For semiconductor nanocrystal cores that emit energy in the visible (e.g., CdS, CdSe, CdTe, ZnSe, ZnTe, GaP, GaAs) or near IR (e.g., InP, InAs, InSb, PbS, PbSe), a material that has a bandgap energy in the ultraviolet regions can be used. Exemplary materials include ZnS, GaN, and magnesium chalcogenides, e.g., MgS, MgSe, and MgTe. For a semiconductor nanocrystal core that emits in the near IR, materials having a bandgap energy in the visible, such as CdS or CdSe, can also be used. The preparation of a coated semiconductor nanocrystal can be found in, e.g., Dabbousi et al. (1997) J. Phys. Chem. B 101:9463) and Kuno et al. (1997) J. Phys. Chem. 106:9869.

Most semiconductor nanocrystals are prepared in coordinating solvent, such as trioctylphosphine oxide (TOPO) and trioctyl phosphine (TOP) resulting in the formation of a passivating organic layer on the nanocrystal surface comprised of the organic solvent. This layer is present on semiconductor nanocrystals containing an overcoating and those that do not contain an overcoating. Thus, either of these classes of passivated semiconductor nanocrystals are readily soluble in organic solvents, such as toluene, chloroform and hexane. These functional moieties can be readily displaced or modified to provide an outer coating that renders the semiconductor nanocrystals suitable for use as the detectable labels of the present invention, as described further below. Furthermore, based upon the desired application, a portion of the semiconductor nanocrystal functionality, or the entire surface of the semiconductor nanocrystal functionality can be modified by a displacement reaction, based upon the desired use therefor.

After selection of the composition of semiconductor nanocrystal for the desired range of spectral emission and selection of a desired surface functionalization compatible with the system of interest, it may also be desirable to select the minimum number of semiconductor nanocrystals needed to observe a distinct and unique spectral emission of sufficient intensity for spectral identification. Selection criteria important in determining the minimum number of semiconductor nanocrystals needed to observe a distinct and unique spectral emission of sufficient intensity include: (1) providing a sufficient number of semiconductor nanocrystals that are bright (i.e., that emit light versus those that are dark) and, (2) providing a sufficient number of semiconductor nanocrystals to average out over the blinking effect observed in single semiconductor nanocrystal emissions. Nirmal et al., (1996) Nature 383:802.

For example, eight or more semiconductor nanocrystals of a particular composition and particle size distribution can be provided. If, for example, the desired method of use utilizes three different particle size distributions of a particular composition, eight of each of the three different particle size distributions of a semiconductor nanocrystal is used, in order to observe sufficiently intense spectral emissions from each to provide reliable information regarding the location or identity of a particular analyte of interest. Fewer than eight semiconductor nanocrystals of a particular composition and particle size distribution can be utilized provided that a unique spectral emission of sufficient intensity is observed, as determined by the selection criteria set forth above.

The above method can be used to prepare separate populations of semiconductor nanocrystals, wherein each population exhibits a different characteristic photoluminescence spectrum. Each of a plurality of populations of semiconductor nanocrystals can be conjugated to distinct first members of binding pairs for use in a multiplexed assay or analytical method in which each of a plurality of corresponding second members of the binding pairs can be detected simultaneously.

The narrow spectral linewidths and nearly gaussian symmetrical lineshapes lacking a tailing region observed for the emission spectra of nanocrystals combined with the tunability of the emission wavelengths of nanocrystals allows high spectral resolution in a system with multiple nanocrystals. In general up to 10-20 or more different-sized nanocrystals or different size distributions of monodisperse populations of nanocrystals from different preparations of nanocrystals, with each sample having a different emission spectrum, can be used simultaneously in one system, i.e., multiplexing, with the overlapping spectra easily resolved using techniques well known in the art, e.g., optically with or without the use of deconvolution software.

As discussed previously, the ability of the semiconductor nanocrystals to produce discrete optical transitions, along with the ability to vary the intensity of these optical transitions, enables the development of a versatile and dense encoding scheme. The characteristic emissions produced by one or more sizes of semiconductor nanocrystals attached to, associated with, or embedded within a particular support, compound or matter enables the identification of the analyte of interest and/or its location. For example, by providing N sizes of semiconductor nanocrystals (each having a discrete optical transition), each having M distinguishable states resulting from the absence of the semiconductor nanocrystal, or from different intensities resulting from a particular discrete optical transition, Mn different states can be uniquely defined. In the case wherein M is 2, in which the two states could be the presence or absence of the semiconductor nanocrystal, the encoding scheme would thus be defined by a base 2 or binary code. In the case wherein M is 3, in which the three states could be the presence of a semiconductor nanocrystal at two distinguishable intensities and its absence, the encoding scheme would be defined by a base 3 code. Herein, such base M codes wherein M is greater than 2 are termed higher order codes. The advantage of higher order codes over a binary order code is that fewer identifiers are required to encode the same quantity of information.

As one of ordinary skill in the art will realize, the ability to develop a higher order encoding system is dependent upon the number of different intensities capable of detection by both the hardware and the software utilized in the decoding system. In particularly preferred embodiments, each discrete emission or color, is capable of being detectable at two to twenty different intensities. In a particularly preferred embodiment wherein ten different intensities are available, it is possible to employ a base 11 code comprising the absence of the semiconductor nanocrystal, or the detection of the semiconductor nanocrystal at 10 different intensities.

Clearly, the advantages of the semiconductor nanocrystals, namely the ability to observe discrete optical transitions at a plurality of intensities, provides a powerful and dense encoding scheme that can be employed in a variety of disciplines. In general, one or more semiconductor nanocrystals may act as a barcode, wherein each of the one or more semiconductor nanocrystals produces a distinct emissions spectrum. These characteristic emissions can be observed as colors, if in the visible region of the spectrum, or may also be decoded to provide information about the particular wavelength at which the discrete transition is observed. Likewise, for semiconductor nanocrystals producing emissions in the infrared or ultraviolet regions, the characteristic wavelengths that the discrete optical transitions occur at provide information about the identity of the particular semiconductor nanocrystal, and hence about the identity of or location of the analyte of interest.

The color of light produced by a particular size, size distribution and/or composition of a semiconductor nanocrystal can be readily calculated or measured by methods which will be apparent to those skilled in the art. As an example of these measurement techniques, the bandgaps for nanocrystals of CdSe of sizes ranging from 12 Å to 115 Å are given in Murray et al. (1993) J. Am. Chem. Soc. 115:8706. These techniques allow ready calculation of an appropriate size, size distribution and/or composition of semiconductor nanocrystals and choice of excitation light source to produce a nanocrystal capable of emitting light device of any desired wavelength.

An example of a specific system for automated detection for use with the present methods includes, but is not limited to, an imaging scheme comprising an excitation source, a monochromator (or any device capable of spectrally resolving the image, or a set of narrow band filters) and a detector array. In one embodiment, the apparatus consists of a blue or UV source of light, of a wavelength shorter than that of the luminescence detected. This may be a broadband UV light source, such as a deuterium lamp with a filter in front; the output of a white light source such as a xenon lamp or a deuterium lamp after passing through a monochromator to extract out the desired wavelengths; or any of a number of continuous wave (cw) gas lasers, including but not limited to any of the Argon Ion laser lines (457, 488, 514, etc. nm), a HeCd laser; solid state diode lasers in the blue such as GaN and GaAs (doubled) based lasers or the doubled or tripled output of YAG or YLF based lasers; or any of the pulsed lasers with output in the blue, to name a few.

The luminescence from the dots may be passed through an imaging subtracting double monochromator (or two single monochromators with the second one reversed from the first), for example, consisting of two gratings or prisms and a slit between the two gratings or prisms. The monochromators or gratings or prisms can also be replaced with a computer controlled color filter wheel where each filter is a narrow band filter centered at the wavelength of emission of one of the dots. The monochromator assembly has more flexibility because any color can be chosen as the center wavelength. Furthermore, a CCD camera or some other two dimensional detector records the images, and software color codes that image to the wavelength chosen above. The system then moves the gratings to a new color and repeats the process. As a result of this process, a set of images of the same spatial region is obtained and each is color-coded to a particular wavelength that is needed to analyze the data rapidly.

In another embodiment, the apparatus is a scanning system as opposed to the above imaging scheme. In a scanning scheme, the sample to be analyzed is scanned with respect to a microscope objective. The luminescence is put through a single monochromator or a grating or prism to spectrally resolve the colors. The detector is a diode array that then records the colors that are emitted at a particular spatial position. The software then ultimately recreates the scanned image and decodes it.

XI. Production of Semiconductor Nanocrystal Conjugates

The present invention utilizes various conjugates that generally comprise a biological molecule and one or more semiconductor nanocrystals, such that the conjugate can detect the presence, absence and/or amounts of various complexes formed on addressable arrays. Without limitation, semiconductor nanocrystal conjugates comprise any molecule or molecular complex, linked to a semiconductor nanocrystal, that can interact with a biological target, to detect biological processes, or reactions, as well as alter biological molecules or processes. Preferably, the molecules or molecular complexes or conjugates physically interact with a biological compound. Preferably, the interactions are specific. The interactions can be, but are not limited to, covalent, noncovalent, hydrophobic, hydrophilic, electrostatic, van der Waals, or magnetic. Preferably, these molecules are small molecules, proteins, or nucleic acids or combinations thereof.

Semiconductor nanocrystal conjugates can be made using techniques known in the art. For example, moieties such as TOPO and TOP, generally used in the production of semiconductor nanocrystals, as well as other moieties, can be readily displaced and replaced with other functional moieties, including, but not limited to carboxylic acids, amines, aldehydes, and styrene to name a few. One of ordinary skill in the art will realize that factors relevant to the success of a particular displacement reaction include the concentration of the replacement moiety, temperature and reactivity. Thus, for the purposes of the present invention, any functional moiety may be utilized that is capable of displacing an existing functional moiety to provide a semiconductor nanocrystal with a modified functionality for a specific use.

The ability to utilize a general displacement reaction to modify selectively the surface functionality of the semiconductor nanocrystals enables functionalization for specific uses. For example, because detection of biological compounds is most preferably carried out in aqueous media, typically the present invention utilizes semiconductor nanocrystals that are solubilized in water. In the case of water-soluble semiconductor nanocrystals, the outer layer includes a compound having at least one linking moiety that attaches to the surface of the particle and that terminates in at least one hydrophilic moiety. The linking and hydrophilic moieties are spanned by a hydrophobic region sufficient to prevent charge transfer across the region. The hydrophobic region also provides a “pseudo-hydrophobic” environment for the nanocrystal and thereby shields it from aqueous surroundings. The hydrophilic moiety can be a polar or charged (positive or negative) group. The polarity or charge of the group provides the necessary hydrophilic interactions with water to provide stable solutions or suspensions of the semiconductor nanocrystal. Exemplary hydrophilic groups include polar groups such as hydroxides (—OH), amines, polyethers, such as polyethylene glycol and the like, as well as charged groups, such as carboxylates (—CO₂⁻), sulfonates (SO₃⁻), phosphates (—PO₄²⁻ and —PO₃²⁻), nitrates, ammonium salts (—NH₄⁺), and the like. A water-solubilizing layer is found at the outer surface of the overcoating layer. Methods for rendering semiconductor nanocrystals water-soluble are known in the art and described in, e.g., PCT Publication No. WO 00/17655, published Mar. 30, 2000.

The affinity for the nanocrystal surface promotes coordination of the linking moiety to the semiconductor nanocrystal outer surface and the moiety with affinity for the aqueous medium stabilizes the semiconductor nanocrystal suspension.

A displacement reaction can be employed to modify the semiconductor nanocrystal to improve the solubility in a particular organic solvent. For example, if it is desired to associate the semiconductor nanocrystals with a particular solvent or liquid, such as pyridine, the surface can be specifically modified with pyridine or pyridine-like moieties to ensure solvation.

The surface layer can also be modified by displacement to render the semiconductor nanocrystal reactive for a particular coupling reaction. For example, displacement of TOPO moieties with a group containing a carboxylic acid moiety enables the reaction of the modified semiconductor nanocrystals with amine containing moieties (commonly found on solid support units) to provide an amide linkage. Additional modifications can also be made such that the semiconductor nanocrystal can be associated with almost any solid support such as those describe supra.

For example, the semiconductor nanocrystals of the present invention can readily be functionalized to create styrene or acrylate moieties, thus enabling the incorporation of the semiconductor nanocrystals onto polystyrene, polyacrylate or other polymers such as polyimide, polyacrylamide, polyethylene, polyvinyl, polydiacetylene, polyphenylene-vinylene, polypeptide, polysaccharide, polysulfone, polypyrrole, polyimidazole, polythiophene, polyether, epoxies, silica glass, silica gel, siloxane, polyphosphate, hydrogel, agarose, cellulose, and the like.

For a detailed description of these linking reactions, see, e.g. U.S. Pat. No. 5,990,479; Bruchez et. al. (1998) Science 281:2013-2016., Chan et. al. (1998) Science 281:2016-2018, Bruchez “Luminescent Semiconductor Nanocrystals: Intermittent Behavior and use as Fluorescent Biological Probes” (1998) Doctoral dissertation, University of Califormia, Berkeley, and Mikulec “Semiconductor Nanocrystal Colloids: Manganese Doped Cadmium Selenide, (Core)Shell Composites for Biological Labeling, and Highly Fluorescent Cadmium Telluride” (1999) Doctoral dissertation, Massachusetts Institute of Technology.

The following examples are provided to illustrate certain aspects of the invention and are not to be interpreted so as to limit the scope of the invention.

### EXAMPLE 1

### Preparation of Detectable Probes For cDNA Array Labeling

cDNA microarray slides were prepared as described in the Fabrication section of www.nhgri.nih.gov/DIR/microarray. Further guidance on fabrication, sample labeling and conditions for hybridization using microarrays is provided, for example, by Bittner M., et al. (2000) Nature 406:536-540; Khan J., et al. (1999) Electrophoresis 20:223-9; Duggan, D.J. (1999) Science 283:83-87; and DeRisi, J. et al. (1996) 14:457-60.

A. cDNA labeled with biotin.

The preparation of RNA, sample labeling by reverse transcription and hybridization were performed by using method as described in Khan et al. (1999) Biochim. Biophys. Acta 1423:17-28 or from www.nhgri.nih.gov/DIR/microarray or other published methods. The cDNA was labeled with biotin. For labeling with semiconductor nanocrystals (SCNC)-streptavidin conjugates, Cy®3 dUTP was replaced with biotin-16-dUTP (Roche Molecular Biochemicals, Indianapolis, Ind.).

After hybridization, the slide was incubated in 4× SSC, 0.1% Tween® 20, 1% bovine serum albumin (BSA) at room temperature for a minimal of 30 minutes and rinsed in 1× phosphate-buffered saline (PBS), 1% BSA, 1 mM MgCl₂. The slide was incubated in 25 nM 630 nm SCNC-streptavidin in 1× PBS, 1% BSA, 10 mM MgCl₂ for 1 hour at room temperature. The slide was rinsed in 1× PBS, 1% BSA, 1 mM MgCl₂ followed by 10 mM Phosphate buffer pH 7.4 and spin-dried in centrifuge at 500 rpm for 5 minutes.

The spots on the microarray were viewed under a fluorescence microscope and scanner optimized for SCNCs.

B. cDNA labeled with biotin and Cy®3 or Cy®5.

The preparation of RNA, sample labeling by reverse transcription and hybridization were performed using methods described in Khan et al. (1999) Biochim. Biophys. Acta 1423:17-28 or from www.nhgri.nih.gov/DIR/microarray. One cDNA was labeled with biotin and the other with Cy3 or Cy5. For labeling with semiconductor nanocrystal (SCNC)-streptavidin, Cy5- or Cy3-dUTP was replaced with biotin-16-dUTP (Roche Molecular Biochemicals, Indianapolis, Ind.).

After the hybridization step, the microarray slide was incubated in 4× SSC, 0.1% Tween 20, 1% BSA for 30-60 minutes at room temperature, rinsed in 0.06× SSC, and spun dry at 500 rpm for 5 minutes in a centrifuge with a horizontal rotor for microplates. SCNC-streptavidin were added to 6× SSPE, 1% BSA, 10 mM MgCl₂ to a final concentration of 25 nM. Depending on the size of the array, 40-80 μL of the SCNC-streptavidin was applied on the array area, a coverslip was added and the covered microarray was incubated in a humidified container for 1 hour at room temperature. The slide was rinsed in 1× SSPE followed by 0.06× SSPE and spin-dried in centrifuge at 500 rpm for 5 minutes.

The microarray was viewed on a fluorescence microscope. SCNC-labeled cDNA hybridized to the microarray was easily detected under fluorescence microscopy.

C. The preparation of RNA, sample labeling by reverse transcription and hybridization are performed by using method as described in Khan et al. (1999) Biochim. Biophys. Acta 1423:17-28 or from web site www.nhgri.nih.gov/DIR/microarray, or by any suitable method known to those skilled in the art. One cDNA is labeled with biotin and the other with a hapten (e.g., fluorescein, digoxygenin, or estradiol). For labeling with SCNC-streptavidin, and SCNC-anti-hapten, Cy5- and Cy3-dUTP is replaced with biotin-16-dUTP (Roche Molecular Biochemicals, Indianapolis, Ind.) and dUTP-hapten (e.g., fluorescein-12-dUTP, DIG 1-dUTP, estradiol-15-dUTP).

After hybridization, the slide is incubated in blocking solution 4× SSC, 0.1% Tween 20,1% BSA for 30-60 minutes at room temperature, rinsed in 0.06× SSC, and dried by spinning at 500 rpm for 5 minutes in a centrifuge with a horizontal rotor for microplates. SCNC-streptavidin and SCNC-anti-hapten are added to 6× SSPE, 1% BSA, 10 mM MgCl₂ to a final concentration of 25 nM each. Depending on the size of the array, 40-80 μL of the mixture is applied on the array area, a coverslip is added over the array area and the covered array is incubated in a humidified container for 1 hour at room temperature. The slide is rinsed in 1× SSPE followed by 0.06× SSPE and dried by spinning in centrifuge at 500 rpm for 5 minutes. The microarray is read on a scanner optimized for SCNC emission.

D. cDNA labeled with SCNC1-dUTP and SCNC2-dUTP

The preparation of RNA, sample labeling by reverse transcription and hybridization are performed by using method as described in Khan et al. (1999) Biochim. Biophys. Acta 1423:17-28 or from web site www.nhgri.nih.gov/DIR/microarray or by other methods well known in the art. One cDNA is labeled with a first SCNC (SCNC1) have a first emission spectrum and the other with a second SCNC (SCNC2) having an emission spectrum distinct from SCNC1. For labeling with SCNC1 and SCNC2, Cy5- or Cy3-dUTP is replaced with SCNC1-dUTP and SCNC2-dUTP, respectively. After hybridization and washes, the dried slide is read on a scanner optimized for SCNCs.

It is anticipated that each distinctly labeled cDNA will be easily distinguished.

### EXAMPLE 2

### cDNA for Oligonucleotide Microarray

Oligonucleotide microarray chips can purchased from Operon Technologies, Inc. or from other sources.

A. cDNA labeled with biotin and Cv®3 or Cy®

The preparation of RNA and sample labeling by reverse transcription are performed using method described in Khan et al. (1999) Biochim. Biophys. Acta 1423:17-28, from web site www.nhgri.nih.gov/DIR/microarrav, or using methods known to those of skill in the art. Alternatively, preparation of cDNA and hybridization can be performed using protocols described in web site www.pangloss.com/seidel/Protocols. One cDNA is labeled with biotin and the other with Cy3 or Cy5. For labeling with SCNC-streptavidin, Cy5- or Cy3-dUTP is replaced with biotin-16-dUTP (Roche Molecular Biochemicals, Indianapolis, Ind.).

The hybridization buffer contains the labeled cDNAs in 4× SSC and 1 mg/ml poly(dA) (Pharmacia) and 0.2 mg /ml yeast tRNA (Sigma). The probe mixture is denatured at 98° C. for 2 minutes, cooled to 45° C. and a small volume of 10% SDS solution is added to a final concentration of 0.2% SDS. The volume of 15-30 μL depending on the size of the array is applied onto microarray area and the microarray area is covered with a glass cover-slip. The covered microarray is placed in a humidified chamber and incubated overnight at 65° C. After hybridization, the slide is sequentially rinsed in 1× SSC with 0.03% SDS, 0.2× SSC and 0.05× SSC. The slide is dried by spinning in a centrifuge with horizontal rotor at 500 rpm for 5 minutes. The slide is incubated in blocking solution 4× SSC, 0.1% Tween 20, 1% BSA for 30-60 minutes at room temperature, rinsed in 0.05× SSC, and dried by spinning in centrifuge. SCNC-streptavidin is added to 6× SSPE, 1% BSA, 10 mM MgCl₂ to a final concentration of 25 nM. Depending on the size of the array, 40-80 μL of the SCNC-streptavidin is applied on the array area, a coverslip is applied over the array area and the cover array is incubated in a humidified container for 1 hour at room temperature. The slide is rinsed in 1× SSPE followed by 0.06× SSPE and dried by spinning in centrifuge at 500 rpm for 5 minutes.

The microarray is read in a scanner optimized for SCNCs and Cy dyes.

B. cDNA labeled with biotin and hapten The preparation of RNA and sample labeling by reverse transcription are performed using method described in Khan et al. (1999) Biochim. Biophys. Acta 1423:17-28 or from web site www.nhgri.nih.gov/DIR/microarray. Alternatively, hybridization can be performed using protocols described in web site www.pangloss.com/seidel/Protocols. One cDNA is labeled with biotin and the other with a hapten such as digoxygenin, fluorescein, estradiol. For labeling with SCNC-streptavidin and SCNC-anti-hapten, Cy5- or Cy3-dUTP is replaced with biotin-16-dUTP (Roche Molecular Biochemicals, Indianapolis, Ind.) and hapten-dUTP (e.g., fluorescein-12-dUTP, DIG 1 1-dUTP, estradiol-15-dUTP).

The hybridization buffer contains the labeled cDNAs in 4× SSC and 1 mg/ml poly(dA) (Pharmacia), 0.2 mg /ml yeast tRNA (Sigma), the probe mixture is denatured at 98° C. for 2 minutes, cool to 45° C. and a small volume of 10% SDS solution is added to a final concentration of 0.2% SDS. The volume 15-30 μL depending on the size of the array is applied on microarray, a cover slip is placed over the array area, and the covered array is placed in a humidified chamber and incubated overnight at 65° C. After hybridization, the slide is sequentially rinsed in 1× SSC with 0.03% SDS, 0.2× SSC and 0.05× SSC. The slide is dried by spinning in centrifuge with horizontal rotor for microplates at 500 rpm for 5 minutes.

The slide is incubated in blocking solution 4× SSC, 0.1% Tween 20, 1% BSA for 30-60 minutes at room temperature, rinsed in 0.05× SSC, and dried by spinning in centrifuge. SCNC-streptavidin and SCNC-anti-hapten is added to 6× SSPE, 1% BSA, 10 mM MgCl₂ to a final concentration of 25 nM each. Depending on the size of the array, 40-80 μL of the mixture is applied on the array area, a cover slip is applied over the array area and the covered array is incubated in a humidified container for 1 hour at room temperature. The slide is rinsed in 1× SSPE followed by 0.06× SSPE and dried by spinning in centrifuge at 500 rpm for 5 minutes.

The microarray is read on a scanner optimized for SCNCs.

C. CDNA labeled with SCNC1-dUTP and SCNC2-dUTP

The preparation of RNA and sample labeling by reverse transcription are performed by using method as described in Khan et al. (1999) Biochim. Biophys. Acta 1423:17-28 or from web site www.nhgri.nih.gov/DIR/microarray. Alternatively, hybridization can be performed using protocols described in web site www.pangloss.com/seidel/Protocols. One cDNA is labeled with SCNC1 and the SCNC2. For labeling cDNA with SCNC1 and SCNC2, Cy5- or Cy3-dUTP is replaced with SCNCl-dUTP and SCNC2-dUTP.

The hybridization buffer contains the labeled cDNAs in 4× SSC and 1 mg/ml poly(dA) (Pharmacia), yeast tRNA 0.2 mg /ml, the probe mixture is denatured at 98° C. for 2 minutes, cooled to 45° C. and a small volume of 10% SDS solution is added to a final concentration of 0.2% SDS. The volume of hybridization mixes various from 15-30 μL depending on the size of the array. The mixture is applied on microarray, a cover slip is applied over the array area, the covered microarray is placed in a humidified chamber and incubated overnight at 65° C. After hybridization, the slide is sequentially rinsed in 1× SSC with 0.03% SDS, 0.2× SSC and 0.05× SSC. The slide is dried by spinning in centrifuge with horizontal rotor for microplates at 500 rpm for 5 minutes.

The microarray is scanned on a scanner optimized for SCNCs.

D. Oligonucleotide microarrays were purchased from Operon Technologies, Inc. These test arrays contained forty spots each, ten spots from each of the four 70 mers selected from Caspase 9-Genbank U56390, Laminin gamma 3 chain precusor, LAMC3-Genbank AF041835, Alpha-tubulin-Genbank K00558 and Ribosomal protein S9-Genbank U14971. 50-mer complementary oligonucleotides biotinylated at the 3′ end were made from each of the four 70 mers.

The hybridization buffer contained the biotin-labeled 50 mer complementary sequences, 4 mg/ml herring sperm DNA as carrier in 4× SSC and 1 mg/ml poly(dA) (Pharmacia), 0.2 mg /ml yeast tRNA (Sigma). The probe mixture was denatured at 98° C. for 2 minutes, cooled to 45° C. and a small volume of 10% SDS solution is added to a final concentration of 0.2% SDS. The volume of 15-30 μL depending on the size of the array was applied on the microarray area and a cover slip was applied over the microarray area. The covered microarray slide was placed in a humidified chamber and incubated overnight at 65° C. After hybridization, the slide was sequentially rinsed in 1× SSC with 0.03% SDS, 0.2× SSC and 0.05× SSC. The slide was dried by spinning in centrifuge with horizontal rotor at 500 rpm for 5 minutes. The slide was incubated in blocking solution 4× SSC, 0.1% Tween 20, 1% BSA for 30-60 minutes at room temperature, rinsed in 0.05× SSC, and dried by spinning in centrifuge. SCNC-streptavidin is added to 6× SSPE, 1% BSA, 10 mM MgCl₂ to a final concentration of 25 nM. Depending on the size of the array, 40-80 μL of the SCNC-streptavidin was applied on the array area and the array area was covered with a glass coverslip. The cover array slide was incubated in a humidified container for 1 hour at room temperature. The slide was rinsed in 1× SSPE followed by 0.06× SSPE and dried by spinning in centrifuge at 500 rpm for 5 minutes.

The microarray was scanned in a scanner optimized for SCNCs.

### EXAMPLE 3

### Protein Array: Detection Using Semiconductor Nanocrystals

A protein array was prepared to interrogate protein species on a spatially addressed array. Protein generated from any source (e.g., a recombinant expression system, a differentially treated cell supernatants, or the like) can be immobilized in a small, spatially addressed spot on a substrate. The spot size can vary from micrometer to millimeter diameter dependent on the assay substrate.

In this Example, 1 μL of rabbit IgG or mouse IgG was spotted onto nitrocellulose and allowed to dry; 50 spots of each different IgG dilution were addressed in a 5×10 array. The nitrocellulose was then blocked by incubation in phosphate-buffered saline (PBS)/1% bovine serum albumin (BSA) for 30 minutes at room temperature. A 1 μg/ml solution of biotinylated anti-rabbit IgG (Vector) was then applied for 30 minutes and the membrane subsequently washed in excess PBS. The array was then exposed to 5 mls of a 25 nM solution of streptavidin-conjugated 630 nm emitting semiconductor nanocrystals in PBS/1% BSA for 30 minutes at room temperature. The membrane was then washed in excess PBS and the luminescence from the semiconductor nanocrystals was detected using an ultraviolet transilluminator (Stratagene Eagle Eye®) and a microarray scanner set to excite at 488 nm with an argon ion laser.

Less than 1 ng of antibody was specifically detected using each detection device.

### EXAMPLE 4

### Tissue Array: Detection Using Semiconductor Nanocrystals

Multiple intracellular markers can be simultaneously analyzed with semiconductor nanocrystal-labeled ligands. This, coupled with a spatial arraying of tissue samples in a defined area, allows a further increase in the throughput of analyzing cellular markers. Small sections of tissue can be immobilized on a microscope slides or some other support as is well known in the art. The tissue source can be derived from a living organism, from a population of cultured cells treated in various ways, or the like.

In this Example, a specific intracellular antigen has been detected on a tissue section attached to a microscope slide. The tissue section was mouse stomach and kidney and was purchased from InovaDX (San Diego, Calif.). The goal of this Example was to detect the presence or absence of auto-immune markers, antinuclear antibodies (ANA), that recognize nuclear antigens. The anti-nuclear antibodies can be specifically detected using a biotinylated anti-human antibody followed by a binding thereto of a streptavidin-conjugated semiconductor nanocrystal.

The tissue section was incubated for one hour with a positive control containing ANA (InovaDX) or with a human serum sample diluted in phosphate-buffered saline (PBS)/1% bovine serum albumin (BSA). A negative control sample was also provided by InovaDX and this is also incubated with a section to provide background or non-specific binding information. The section was then washed by repeated immersion in PBS. The section was then incubated with 3 μg/ml biotinylated anti-human antibody (Vector) for a further 30 minutes at room temperature. The slide was washed in PBS. Finally, the section was incubated with streptavidin-conjugated semiconductor nanocrystals (both 525 nm and 630 run emitting nanocrystals have been used) for 30 minutes at room temperature and finally washed in PBS. A solution of 50% glycerol in PBS was used to mount a coverslip and the section is examined under an epi-fluorescent microscope.

The nuclei were clearly observed as brightly stained whereas the cytosol and surrounding tissue were not stained. No nuclei were observed in the negative control section.

### EXAMPLE 5

### Single Target Counting

The goal of these studies was to demonstrate that single analyte targets can be detected and quantified. 10 μg/ml of rabbit IgG diluted in PBS was passively adsorbed to the surface of standard glass coverslips. Excess antibody was removed and the surfaces were blocked with bovine serum albumin (3% BSA in PBS overnight at 4° C. or 2 hours at room temperature). Each coverslip was immersed in different concentrations of biotinylated anti-rabbit IgG (10 nM to 100 fM in PBS/1% BSA). After incubating for 15 minutes at room temperature, the cover slips were washed in excess PBS and incubated with 10 nM streptavidin functionalized semiconductor nanocrystals (580 nm emission) at room temperature for 10 minutes. After 30 minutes of washing in PBS/1%BSA/0.1% Igepal® at room temperature, samples were imaged with a fluorescence microscope. Signals from single bound analyte molecules and the density of molecules decreased as a function of analyte concentration.

The results were quantified by counting analyte molecules in a defined area of the assay surface (a circular region of about 60 μm in diameter defined by the illumination pattern of our single molecule microscope). The results were linear with concentration of biotinylated rabbit IgG and the sensitivity extended to densities of about 0.001 molecules/μm².

It is understood that the examples and embodiments described herein are for illustrative purposes only and that various modifications or changes in light thereof will be suggested to persons skilled in the art and are to be included within the spirit and purview of this application and scope of the appended claims. All publications, patents and patent applications cited herein are hereby incorporated by reference in their entirety for all purposes to the same extent as if each individual publication, patent or patent application were specifically and individually indicated to be so incorporated by reference.

What is claimed is:

1. An analytical method of detecting a ligand of interest in a sample, comprising:
(a) providing a first plurality of antiligands immobilized on a solid support at positionally distinct locations thereon to provide a first array, wherein the plurality of antiligands comprises a first antiligand capable of binding specifically to a first ligand of interest;
(b) contacting the array with a sample containing or suspected of containing the first ligand, wherein the first ligand is linked through a linker to a first semiconductor nanocrystal before, during or after the contacting, under conditions in which the first ligand binds specifically to the first antiligand to form a first complex;
(c) optionally, removing unbound ligand from the array; and
(d) identifying the location of the first complex by detecting and, optionally, quantifying the presence in the first complex of the first semiconductor nanocrystal.

2. The method of claim 1, wherein the linker comprises two members of a binding pair, a first member attached to the first ligand and a second member attached to the first semiconductor nanocrystal.

3. The method of claim 1, wherein
(a) the sample contains a second ligand linked to a detectably distinct second semiconductor nanocrystal, wherein the second ligand is capable of binding specifically to a second immobilized antiligand to form a second complex; and
(b) identifying comprises determining which location or locations of the array include the first complex, the second complex or the first and second complexes by detecting and, optionally, quantifying simultaneously or sequentially the presence in the first and second complexes of the first and second semiconductor nanocrystals.

4. The method of claim 1, wherein the antiligands are nucleic acid probes and the first ligand is a target nucleic acid.

5. The method of claim 3, wherein the antiligands are nucleic acid probes and the first and second ligands are target nucleic acids.

6. The method of claim 4, wherein the first ligand is linked to the first semiconductor nanocrystal prior to the contacting step.

7. The method of claim 5, wherein the first and second ligands are linked to the first and second semiconductor nanocrystals prior to the contacting step.

8. The method of claim 6, wherein the first ligand bears a single first semiconductor nanocrystal.

9. The method of claim 7, wherein the first ligand and the second ligand bear a single first and a single second semiconductor nanocrystal, respectively.

10. The method of claim 4, wherein the linker comprises two members of a binding pair, a first member coupled to the target nucleic acid and the second member coupled to the semiconductor nanocrystal.

11. The method of claim 4, further comprising:
(e) providing a second plurality of antiligands immobilized on a solid support at positionally distinct locations thereon to provide a second array, wherein the second plurality of antiligands comprises a second antiligand capable of binding specifically to a first ligand of interest and wherein each location comprises an antiligand capable of binding to a distinct ligand of interest
(f) repeating steps (b), (c), and (d) with the second array; and
(g) comparing which nucleic acid probes from the two arrays are bound to the first ligand.

12. The method of claim 5, further comprising
(e) providing a second plurality of antiligands immobilized on a solid support at positionally distinct locations thereon to provide a second array, wherein the second plurality of antiligands comprises a second antiligand capable of binding specifically to a first ligand of interest and wherein each location comprises an antiligand capable of binding to a distinct ligand of interest
(f) repeating steps (b), (c) and (d); and
(g) comparing which nucleic acid probes from the first and second arrays are bound to the first and second ligands.

13. The method of claim 4, wherein the nucleic acid probes are allele-specific nucleic acid probes.

14. The method of claim 13, wherein
(a) the sample contains or is suspected of containing a target nucleic acid that has a first allelic site and a second allelic site;
(b) the plurality of probes includes a set of capture probes, each capture probe being capable of forming a hybridization complex with a distinct allelic form of the target nucleic acid at the first allelic site, whereby if the sample contains the target nucleic acid, then the capture probe complementary to the nucleotide at the first allelic site forms a stable hybridization complex with the target nucleic acid; and
(c) identifying comprises determining which of the capture probes are bound to the target nucleic acid.

15. The method of claim 14, further comprising contacting the target nucleic acids within hybridization complexes with a pool of nucleic acid detection probes, detection probes within the pool comprising a detectably distinct semiconductor nanocrystal and able to form a stable hybridization complex with a different allelic form of the target nucleic acid at the second allelic site, whereby a detection probe from the pool that is complementary to the nucleotide at the second allelic site forms a stable hybridization complex with the target nucleic acid; and wherein identifying comprises determining which of the detection probes is bound to the target nucleic acid.

16. The method of claim 1, wherein the plurality of antiligands are proteins.

17. The method of claim 16, wherein the ligand is a protein.

18. The method of claim 16, wherein the antiligands are antibodies.

19. The method of claim 16, wherein
(a) the sample contains a second ligand linked to a detectably distinct second semiconductor nanocrystal that is capable of binding specifically to a second immobilized antiligand to form a second complex; and
(b) identifying comprises determining which location or locations of the array include the first complex, the second complex or the first and second complexes by detecting and, optionally, quantifying simultaneously or sequentially the presence in the first and second complexes of the first and second semiconductor nanocrystals.

20. The method of claim 1, wherein the antiligand is a component of a tissue specimen.

21. The method of claim 20, wherein
(a) the sample contains a second ligand linked to a detectably distinct second semiconductor nanocrystal that is capable of binding specifically to a second immobilized antiligand to form a second complex; and
(b) identifying comprises determining which location or locations of the array include the first complex, the second complex or the first and second complexes by detecting and, optionally, quantifying simultaneously or sequentially the presence in the first and second complexes of the first and second semiconductor nanocrystals.

22. The method of claim 20, wherein the antiligands are selected from the group consisting of proteins, nucleic acid targets, oligosaccharides and combinations thereof, and the ligands are independently selected from the group consisting of antibodies, nucleic acid probes, lectins, aptamers and combinations thereof.

23. The method of claim 22, wherein the antiligands are distinct target nucleic acids and the ligands are nucleic acid probes.

24. The method of claim 22, wherein the antiligands are proteins and the ligands are proteins.

25. The method of claim 1, wherein the plurality of antiligands is an aptamer.

26. The method of claim 1, wherein the ligand is an aptamer.

27. An analytical method, comprising:
(a) providing a first plurality of nucleic acid primers having a 3′ end and a 5′ end and which primers are immobilized on a solid support at positionally distinct locations thereon to provide a first array, wherein the plurality comprises a first primer complementary to a first target nucleic acid having an allelic site;
(b) contacting the first array with a sample containing or suspected of containing the first target nucleic acid, in the presence of a first terminating nucleotide linked to a first semiconductor nanocrystal through a linker, under conditions such that the first target nucleic acid hybridizes to the first primer to form a first target-primer complex and such that if the first terminating nucleotide is complementary to the nucleotide at the allelic site the first primer is extended to incorporate the first terminating nucleotide to provide an extended primer; and
(c) identifying which location or locations includes extended primer by detecting the presence therein of the first semiconductor nanocrystal.

28. The method of claim 27, wherein the 3′ end of the primer hybridizes immediately adjacent the allelic site.

29. The method of claim 27, wherein the linker comprises two members of a first binding pair, the first member attached to the first terminating nucleotide and the second member attached to the first semiconductor nanocrystal.

30. The method of claim 27, wherein the first terminating nucleotide is a dideoxynucleotide.

31. The method of claim 27, wherein step (b) comprises contacting the first array with a sample containing or suspected of containing the first target nucleic acid, in the presence of the first terminating nucleotide, a second terminating nucleotide, a third terminating nucleotide and a fourth terminating nucleotide, wherein the first, second, third and fourth terminating nucleotides are linked to detectably distinct first, second, third and fourth semiconductor nanocrystals, respectively, through a linker, under conditions such that the first target nucleic acid hybridizes to the first primer to form a first target-primer complex and such that if the first, second, third, or fourth terminating nucleotide is complementary to the nucleotide at the allelic site the first primer is extended to incorporate the first, second, third or fourth terminating nucleotide to provide an extended primer, and step (d) comprises identifying which location or locations includes extended primer by detecting the presence therein of the first, second, third or fourth semiconductor nanocrystal.

32. An analytical method, comprising:
(a) providing a first plurality of antiligands immobilized on a solid support at positionally distinct locations thereon to provide a first array, wherein the first plurality comprises a first antiligand that is a binding partner of a first ligand;
(b) contacting the first array with a sample containing or suspected of containing the first ligand, whereby the first antiligand and the first ligand interact to form a first binary complex;
(c) contacting the first binary complexes with a second antiligand wherein the second antiligand is (i) a binding partner of the first ligand and (ii) linked to a first semiconductor nanocrystal through a linker, whereby the second antiligand binds to the first ligand in the first binary complex to form a first ternary complex; and
(d) identifying which location of the array includes the first ternary complex by detecting the presence therein of the first semiconductor nanocrystal.

33. The method of claim 32, wherein the first ligand is a protein.

34. The method of claim 32, wherein the first ligand is a nucleic acid.

35. The method of claim 32, wherein the first and second antiligands are first and second antibodies, respectively.

36. The method of claim 32, wherein the first and second antiligands are first and second nucleic acid probes, respectively.

37. The method of claim 32, wherein the first ligand is linked to a second semiconductor nanocrystal through a linker and step (d) comprises determining which location of the array includes the first binary complex by detecting the presence therein of the second semiconductor nanocrystal.

38. The method of claim 37, wherein the second semiconductor nanocrystal and the first semiconductor nanocrystal are detectably distinct.

39. The method of claim 38, wherein the first and second antiligands are first and second antibodies.

40. An analytical method, comprising:
(a) providing a first plurality of antiligands immobilized on a solid support at positionally distinct locations thereon to provide a first array, wherein the plurality comprises a first antiligand that is a binding partner of a first ligand;
(b) contacting the first array with a sample containing or suspected of the first ligand, whereby the first ligand and the first antiligand interact to form a first complex;
(c) labeling the first ligand in the first complex with a first semiconductor nanocrystal; and
(d) identifying which location of the array includes the first complex by detecting the presence therein of the first semiconductor nanocrystal.

41. The method of claim 40, wherein:
(i) the first plurality of antiligands comprises a second antiligand that is a binding partner of a second ligand;
(ii) the sample contains or is suspected of containing the second ligand such that the second ligand and the second antiligand form a second complex;
(iii) step (c) comprises labeling the second ligand in the second complex with a second semiconductor nanocrystal that is detectably distinct from the first semiconductor nanocrystal; and
(iv) step (d) comprises determining which location or locations of the array include the first complex, the second complex or both the first and second complexes by detecting the presence therein of the first and second semiconductor nanocrystals.

42. The method of claim 40, wherein the first ligand comprises a first member of a first binding pair and the semiconductor nanocrystal is linked to a second member of the first binding pair by through a linker.

43. The method of claim 41, wherein the second ligand comprises a first member of a second binding pair and the second semiconductor nanocrystal is linked to a second member of the second binding pair.
